(19) United States
(12) Patent Application Publication
(10) Pub. No.: US 2024/0252429 A1
(43) Pub. Date: Aug. 1, 2024

(54) EXTENDED-WEAR SILICONE HYDROGEL CONTACT LENSES AND USES THEREOF

(71) Applicant: OCUMEDIC, INC., Mullica Hill, NJ (US)

(72) Inventors: Mark E. Byrne, Mullica Hill, NJ (US); Stephen A. DiPasquale, Mullica Hill, NJ (US)

(21) Appl. No.: 18/560,787

(22) PCT Filed: May 13, 2022
(86) PCT No.: PCT/US2022/029256

§ 371 Date: Nov. 14, 2023

Related U.S. Application Data
(60) Provisional application No. 63/201,838, filed on May 14, 2021.

(51) Int. Cl. A61K 9/00; A61K 45/06; A61K 47/34; A61P 27/02; G02C 7/04

(52) U.S. Cl. CPC A61K 9/0051; A61K 45/06; A61K 47/34; A61P 27/02; G02C 7/041

(57) ABSTRACT

Contact lens delivery systems containing ocular therapeutic agent(s) within a crosslinked polymeric silicone hydrogel matrix with macromolecular memory sites to release the ocular therapeutic agent(s) from the hydrogel matrix over time are provided. Methods of treatment by contacting the contact lens delivery systems to one or both eyes of a mammal to provide a therapeutically optimal timed release of ocular therapeutic agent(s) within the contact lens delivery system are also provided.

### CROSS REFERENCE TO RELATED APPLICATIONS

This application claims priority under 35 U.S.C. § 119 to provisional patent application U.S. Ser. No. 63/201,838, filed May 14, 2021. The provisional patent application is herein incorporated by reference in its entirety, including without limitation, the specification, claims, and abstract, as well as any figures, tables, appendices, or drawings thereof.

### TECHNICAL FIELD

The disclosure relates generally to silicone hydrogel contact lens delivery systems containing ocular therapeutic agent(s) within a cross-linked polymeric hydrogel matrix with macromolecular memory sites to release the ocular therapeutic agent(s) from the hydrogel matrix over time. The disclosure further relates to use of the silicone hydrogel contact lens delivery systems for treating one or both eyes of a mammal with the ocular therapeutic agent(s). Beneficially, the treatment methods provide efficient and effective therapeutic treatment with optimal time based on clinical evaluation and experience in treating various diseases, disorders or conditions as well as corneal and ocular health.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

The impact of eye disease is far-reaching and directly influences human health and quality of life. Over 1 billion people worldwide have preventable or treatable vision impairment and this number is projected to double by 2050. In 2021, the Lancet Global Health Commission estimated over $400 billion in lost economic productivity due to vision impairment. Therefore, a major scale-up of investment in eye health—including novel pharmaceutics, innovative treatments, and accessibility to services—is needed to increase quality of care, improve outcomes, and meet future needs.

The standard of care in the delivery of ocular therapeutics are topical formulations in the forms of solutions, suspensions, and ointments, which account for over 90% of ophthalmological formulations currently in the market. Poor compliance is a major issue for solutions and suspensions due to missed doses and variability of dose and drop volume related to bottle angle, squeeze pressure, and poorly mixed, unshaken suspensions. Even with perfect compliance (i.e., drops are administered correctly and taken when scheduled), poor bioavailability due to quick tear turnover and ocular transport barriers significantly limit topical eye drop effectiveness. Approximately only 1-8% of the applied therapeutic is able to penetrate the eye, with the remaining 92% entering systemic circulation. Techniques used to increase bioavailability such as increased drug concentration, increased administration frequency, altered drug corneal penetration capability or lipophilicity necessitating shaking the bottle for distribution, increasing formulation viscosity to increase drug residence time, etc., typically lead to sometimes less but usually more difficult patient compliance.

Despite being an easy site to access, drug delivery to the eye is challenging due to presence of blood ocular barriers that impede transport of drugs from the bloodstream and quick tear turnover that greatly reduces residence time of drugs within the tear fluid. These properties significantly increase the difficulty in designing new technologies to treat the eye, necessitating that technologies overcome the natural barriers and low residence time of drugs within the eye without affecting a patient's vision and comfort while being non-invasive with high patient compliance. Over 60 years ago, the discovery of hydrogels for developing the first soft contact lenses was achieved. Since inception, the hydrogel contact lens is one of the most prevalent medical devices with a strong history of success and safety, and currently used by millions of patients worldwide. Although the early technology envisioned the soft contact lens to be a vehicle for delivery of therapeutics to the eye, due to their non-invasive nature and ability to load drugs in the aqueous regions of the lens, no drug delivering contact lens has yet made it to market or been sold commercially. There remains a need in the art for improved contact lens delivery systems.

It is therefore an object of this disclosure to provide silicone hydrogel contact lens delivery systems containing ocular therapeutic agent(s) within a cross-linked polymeric hydrogel matrix with macromolecular memory sites to release the ocular therapeutic agent(s) from the hydrogel matrix over time.

It is an object of the disclosure to provide silicone hydrogel contact lens delivery systems with methods of loading and release that control and extend therapeutic release duration, maintain necessary physical properties of a contact lens, and deliver a therapeutically relevant amount of ocular therapeutic agent(s).

It is an object of the disclosure to achieve sustained in vivo sustained release, such as week-long sustained release, of ocular therapeutic agent(s) from the silicone hydrogel contact lens delivery systems.

It is a further object of the disclosure to provide methods of treating one or both eyes of a mammal in need thereof with the silicone hydrogel contact lens delivery systems. In an object the methods provider an improvement over state of the art drop administration for clinical treatment, such as post-cataract, uveitis and corneal inflammation, pain, infection, post-LASIK, corneal abrasion treatment, and the like.

Other objects, embodiments and advantages of this disclosure will be apparent to one skilled in the art in view of the following disclosure, the drawings, and the appended claims.

### BRIEF SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part. It is a primary object, feature, and/or advantage of the present disclosure to improve on or overcome the deficiencies in the art.

It is an object, feature, and/or advantage of the present disclosure to provide contact lens delivery systems containing ocular therapeutic agents, comprising: a silicone hydrogel contact lens comprising a cross-linked polymeric hydrogel matrix comprising functional monomers and low molecular weight crosslinking agents, and wherein the cross-linked polymeric hydrogel matrix has macromolecular memory sites that complex an ocular therapeutic agent and release the ocular therapeutic agent from the hydrogel matrix over time while in contact with a surface of an eye, wherein the cross-linked polymeric hydrogel matrix contains an effective amount of the at least one ocular therapeutic agent, and wherein the silicone hydrogel contact lens are afocal, multi-focal, vision correcting or non-correcting, plano, or bandage lenses having no vision correction and having an elastic modulus between about 0.5 mPa and about 2.0 MPa.

It is a further object, feature, and/or advantage of the present disclosure to provide methods of treating one or both eyes of a mammal in need thereof, comprising: contacting the contact lens delivery system according to any one of claims 1-15 to one or both eyes of a mammal to provide controlled release of the at least one ocular therapeutic agent for a duration of treatment.

In any of the embodiments, the methods can include wherein the one or both eyes require treatment with steroidal anti-inflammatory drugs (SAIDs) and/or non-steroidal anti-inflammatory drugs (NSAIDs). In any of the embodiments, the methods can include wherein the one or both eyes require antibiotic drug therapy.

In any of the embodiments, the methods can include the contact lens contacting the one or both eyes continuously for a period of less than about 30 days, and/or replaced every about 5 days to about 16 days. In any of the embodiments, the methods can include a duration of treatment is for a period of between about 1 week to about 15 weeks, and/or wherein the contact lens are replaced every about 5 to about 10 days throughout the duration of treatment. In any of the embodiments, the methods can include the effective amount of the at least one ocular therapeutic agent being increased or decreased when the contact lens are replaced.

In any of the embodiments, the methods can include the contact lens used for the treatment of a condition including post-cataract surgery, post-laser-assisted in situ keratomileusis (LASIK) or other forms of laser-assisted ocular and/or vision surgery, uveitis, and corneal abrasion. In embodiments, the other forms of laser-assisted ocular and/or vision surgery can include photorefractive keratectomy (PRK) surgery, small incision lenticule extraction (SMILE) laser surgery, epithelial-LASIK surgery, lens replacement surgery or refractive lens exchange, laser cataract surgery, laser epithelial keratomileusis (LASEK) surgery, and Presby LASIK or multifocal LASIK surgery.

In any of the embodiments, the therapeutic ocular agent in the contact lens delivery system can comprise a drug, such as an antibiotic, an anti-inflammatory, an antihistamine, an antiviral agent, a cancer drug, an anesthetic, a cycloplegic, an anticholinergic, an antimuscarinic, a mydriatics, a lubricant agent, a hydrophilic agent, a decongestant, a vasoconstrictor, vasodilator, an immuno-suppressant, an immuno-modulating agent, an anti-glaucoma agent, an anti-infective, hyperosmolar agent, vitamins, growth factors, growth factor antagonists, sympathomimetics, an adrenergic agonist, an anti-cataract agent, an anti-hypertensive agent, an anti-macular degeneration agent, an ocular permeation enhancing agent, an anti-retinal disease agent, an anti-retinitis pigmentosa agent, an anti-diabetic retinopathy agent, an ocular myopia controlling agent, an ocular diagnostic agent, or combinations thereof. In preferred embodiments, the drug is an anti-inflammatory agent comprising triamcinolone acetonide, dexamethasone, dexamethasone sodium phosphate, and other corticosteroids, bromfenac sodium, diclofenac sodium, and/or non-steroidal anti-inflammatory drugs (NSAIDs). In further preferred embodiments, the drug is an antibiotic comprising moxifloxacin or other quinolone or fluoroquinolone antibiotics, cefuroxime and other cephalosporin antibiotics, vancomycin or other glycopeptide antibiotics, or combinations thereof.

In any of the embodiments, the methods can further include the one or both eyes further treated with an antibiotic with an optional long-acting SAID via intracameral irrigation or injection, subconjunctival or sub-Tenon's injection, or intravitreal injection and/or depot placement prior to the contacting of the contact lens delivery system to the one or both eyes of the mammal.

In any of the embodiments, beneficially the bioavailability of the contact lens delivery system exceeds that of a conventional eye drop therapy for the same indication of use, and wherein preferably the bioavailability of the contact lens delivery system is at least 5 times greater, at least 10 times greater, at least 15 times greater, or at least 20 times greater than a conventional eye drop therapy for the same indication of use. In any of the embodiments, the contact lens delivery system provides a bioavailability in tear film (AUC₀₋₈ days) of at least about 140 μg week/mL, or at least about 1,000 μg week/mL when provided at a Cₘₐₓ concentration of at least about 200 μg/mL. In any of the embodiments, the contact lens delivery system provides a bioavailability in tear film (AUC_{0-24 hours}) of at least about 20 μg day/mL, or at least about 100 μg day/mL when provided at a Cₘₐₓ concentration of at least about 200 μg/mL. In any of the embodiments, the contact lens delivery system provides an average concentration of the at least one ocular therapeutic agent to the eye(s) of at least about 0.005 μg/mL per day, at least about 0.01 μg/mL per day, at least about 0.1 μg/mL per day, at least about 1 μg/mL per day, at least about 10 μg/mL per day, or at least about 100 μg/mL per day based on concentration or tissue density. Moreover, in any of the embodiments, the contact lens delivery system provides an average ocular tissue (e.g., cornea, sclera, choroid, iris/ciliary body, etc.) or aqueous humor concentration of the at least one ocular therapeutic agent of at least about 0.01 μg/mL per day, at least about 0.1 μg/mL per day, at least about 1 μg/mL per day, at least about 10 μg/mL per day, or at least about 100 μg/mL per day.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1A is an illustration of macromolecular memory for bromfenac loading and controlled release in a contact lens.

FIG. 1B is an illustration of a control lens where bromfenac is transported from the control lens.

FIG. 1C is an illustration of macromolecular memory for increased bromfenac loading and controlled release, showing an average mesh size of gel (arrow) that has a synergistic effect on drug loading and release with polymer having mobility.

FIG. 2 is a graph depicting the equilibrium mass binding of DS, DMSP and BS in lenses synthesized using the templating process and controls.

FIG. 3A is a top view illustration of a physiological flow microfluidic device.

FIG. 3B is a side view illustration of a physiological flow microfluidic device.

FIG. 4A is a graph depicting the release of bromfenac sodium from templated contact lenses synthesized at different M/T ratios.

FIG. 4B is a graph depicting the controlled dual release of DMSP and DS from templated lenses and control lenses.

FIG. 4C is a graph depicting the dual release of bromfenac sodium and moxifloxacin from templated lenses and control lenses.

FIG. 5A is a graph depicting the optical transmittance of templated BS+MOX loaded lenses.

FIG. 5B is a graph depicting the optical transmittance of templated DS+DMSP loaded lenses.

FIG. 6A is a graph depicting the equilibrium weight swelling ratio and equilibrium volume swelling ratio of lenses templated with BS+MOX and DS+DMSP.

FIG. 6B is a graph depicting the molecular weight between crosslinks and mesh size of lenses templated with BS+MOX and DS+DMSP.

FIG. 7A is a graph depicting the equilibrium binding of bromfenac sodium in Bromfenac Extended-Release Contact Lenses (BERCLs) at different M/T ratios.

FIG. 7B is a graph depicting the microfluidic physiological flow release of bromfenac sodium in PBS from BERCLs synthesized at different M/T ratios and control lens.

FIG. 8A is a graph depicting the equilibrium volume swelling ratio and equilibrium weight swelling ratio of BERCLs and control lenses.

FIG. 8B is a graph depicting average molecular weight between crosslinks and average mesh size of BERCLs and control lenses.

FIG. 8C is a graph depicting the optical transmittance of BERCLs in ALF and PBS.

FIG. 8D is an image showing a BERCL lens.

FIG. 9A is a graph depicting the in vivo release profile of bromfenac from BERCLs in White New Zealand rabbits for 8 days.

FIG. 9B is a graph depicting the expanded Bromday™ topical eye drop concentration profile.

FIG. 10A is a set of images showing histological analysis of ocular tissue treated with BERCLs compared to control tissue with no lens or drugs.

FIG. 10B is a set of images showing TUNEL assay analysis of ocular tissue treated with BERCLs compared to control tissue with no lens or drugs.

Various embodiments of the present disclosure will be described in detail with reference to the drawings, wherein like reference numerals represent like parts throughout the several views. Reference to various embodiments does not limit the scope of the invention. Figures represented herein are not limitations to the various embodiments according to the disclosure and are presented for exemplary illustration of the embodiments of the invention. An artisan of ordinary skill in the art need not view, within isolated figure(s), the near infinite number of distinct permutations of features described in the following detailed description to facilitate an understanding of the embodiments of the present invention.

### DETAILED DESCRIPTION

The present disclosure is not to be limited to that described herein, which can vary and are understood by skilled artisans. No features shown or described are essential to permit basic operation of the present invention unless otherwise indicated. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of the embodiments of the disclosure are presented in a range format. It should be understood that the description in range format is merely for convenience and brevity and should not be construed as an inflexible limitation on the scope of the embodiments of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions. This applies regardless of the breadth of the range.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

Unless defined otherwise, all technical and scientific terms used above have the same meaning as commonly understood by one of ordinary skill in the art to which embodiments of the present disclosure pertain.

The terms “invention” or “present invention” are not intended to refer to any single embodiment of the particular invention but encompass all possible embodiments as described in the specification and the claims.

The term “about,” as used herein, refers to variation in the numerical quantity that can occur, for example, through typical measuring techniques and equipment, with respect to any quantifiable variable, including, but not limited to, mass, volume, time, temperature, pH, and the like. The term “about” also encompasses these variations. Whether or not modified by the term “about,” the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

As used herein, the term “exemplary” refers to an example, an instance, or an illustration, and does not indicate a most preferred embodiment unless otherwise stated.

As used herein, the term “extended-wear” in referring to contact lenses provide silicone hydrogel contact lenses that are suitable for wearing overnight and for multiple days/night continuously.

The term “eye drops” herein is meant to refer to all topological medications administered to a surface of the eye including but not limited to solutions, suspensions, ointments and combination thereof.

The term “generally” encompasses both “about” and “substantially.”

As used herein the term “polymer” refers to a molecular complex comprised of a more than ten monomeric units and generally includes, but is not limited to, homopolymers, copolymers, such as for example, block, graft, random and alternating copolymers, terpolymers, and higher “x”mers, further including their analogs, derivatives, combinations, and blends thereof. Furthermore, unless otherwise specifically limited, the term “polymer” shall include all possible isomeric configurations of the molecule, including, but are not limited to isotactic, syndiotactic and random symmetries, and combinations thereof. Furthermore, unless otherwise specifically limited, the term “polymer” shall include all possible geometrical configurations of the molecule.

The “scope” of the present embodiments of the disclosure is defined by the appended claims, along with the full scope of equivalents to which such claims are entitled. The scope of the embodiments of the disclosure is further qualified as including any possible modification to any of the aspects and/or embodiments disclosed herein which would result in other embodiments, combinations, subcombinations, or the like that would be obvious to those skilled in the art.

The term “substantially” refers to a great or significant extent. “Substantially” can thus refer to a plurality, majority, and/or a supermajority of said quantifiable variable, given proper context.

As used herein, the terms “treat”, “treatment”, “treating” or like terms when used with respect to a disease, disorder, condition or post-surgery or procedure, such as for example, post-cataract surgery, post-laser-assisted in situ keratomileusis (LASIK) or other forms of laser-assisted ocular and/or vision surgery, uveitis, corneal abrasion, etc., refers to a therapeutic or prophylactic treatment that increases the resistance of a subject to development of the disease, disorder or condition, that decreases the likelihood that the subject will develop the disease, disorder or condition, that increases the ability of a subject that has developed the disease, disorder or condition to fight the disease, disorder or condition (e.g., reduce or eliminate at least one symptom typically associated therewith) or prevent the disease, disorder or condition from becoming worse, or that decreases, reduces, or inhibits at least one characteristic or symptom of the disease, disorder or condition thereof by at least about 10% (e.g., at least about 20%, 30%, 40%, 50%, 60%, 70%, 80%, 90%, 95%, or 100%).

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent.” “wt-%,” etc.

### Contact Lens Delivery System

Contact lens delivery systems containing ocular therapeutic agents are provided herein and comprise: a silicone hydrogel contact lens comprising vinyl, bifunctional or multi-functional monomers, oligomers, and/or macromers (collectively referred to herein as functional monomers) and low molecular weight crosslinking agents, and wherein the cross-linked polymeric hydrogel matrix has macromolecular memory sites that complex an ocular therapeutic agent and release the ocular therapeutic agent from the hydrogel matrix over time while in contact with a surface of an eye, wherein the cross-linked polymeric hydrogel matrix is formed by the steps of generating a solution comprising amounts of the ocular therapeutic agent and functional monomers, complexing the functional monomers and the ocular therapeutic agent through non-covalent interactions, initiating copolymerization of the functional monomers and the crosslinking agents, and loading the ocular therapeutic agent into the memory site, and wherein the cross-linked polymeric hydrogel matrix contains an effective amount of the at least one ocular therapeutic agent.

As described herein the silicone hydrogel contact lens comprising the cross-linked polymeric hydrogel matrix with macromolecular memory sites are formed through the copolymerization of the functional monomers and the low molecular weight crosslinking agents. The cross-linked polymeric hydrogel matrix can be formed or fashioned into the desired shape of the silicone hydrogel contact lens. For example, the polymeric hydrogel matrix can be polymerized in a mold or compression casting.

The silicone hydrogel contact lens formed can be afocal, multi-focal, vision correcting or non-correcting, plano, or bandage lenses having no vision correction.

The cross-linked polymeric hydrogel matrix is a silicone hydrogel contact lens, namely any type of silicone hydrogel contact lens including afocal, multi-focal, vision correcting or non-correcting, plano, or bandage lenses having no vision correction. Hydrogels are insoluble, cross-linked (chemically and/or physically crosslinked) polymer network structures composed of hydrophilic homo- or hetero-co-polymers, and have the ability to absorb significant amounts of water. Due to their significant water content, hydrogels also possess a degree of flexibility very similar to natural tissue, which minimizes potential irritation to surrounding membranes and tissues. Various hydrogels with ranges of swellability are used in biomedical and pharmaceutical applications, and drug release therefrom depends on simultaneous rate processes of water migration into the network and drug diffusion outward through the swollen hydrogel. In some embodiments, hydrogels as described herein can further have surface coatings to enhance surface hydrophilicity.

The cross-linked polymeric hydrogel matrix comprise functional monomers. As referred to herein functional monomers include vinyl, bifunctional or multi-functional monomers, oligomers, and/or macromers, including silicone and/or carbon-based polymers or functionalized monomers, oligomers and/or macromers, including organic-based monomers, oligomers and/or macromers. In embodiments the cross-linked polymeric hydrogel matrix comprise functional monomers in addition to other monomers to form the hydrogel matrix.

In some embodiments the functional monomers are added at various monomer to template (M/T) ratios (i.e. functional monomer to the template drug) up to about 10 mol % of total silicone hydrogel matrix.

Exemplary functional monomers and other monomers include N,N-dimethylacrylamide (DMA), 2-hydroxy ethylmethacrylate (HEMA), hydroxyethyl acrylate, hydroxypropyl acrylate, hydroxypropyl methacrylate (HPMA), trimethylammonium 2-hydroxy propylmethacry late hydrochloride, dimethylaminoethyl methacrylate (DMAEM), diethyl aminoethyl methacrylate (DEAEM), diallyl dimethyl ammonium chloride (DADMAC), methacrylamidopropyltrimethyl ammonium chloride (MAPTAC), dimethylaminoethyl-methacrylamide, acrylic acid, methacrylic acid, acrylamide, methacrylamide, allyl alcohol, vinylpyridine, glycerol methacrylate, N-(1,1 dimethyl-3-oxobutyl)acrylamide, N-vinyl-2-pyrrolidone (NVP), acrylic acid, methacrylic acid, N-vinyl piperidone, N-vinyl caprolactam, N-vinyl-N-methyl acetamide, N-vinyl formamide, N-vinyl acetamide, N-vinyl-N-methyl acetamide, N-vinyl-N-ethyl acetamide, N-vinyl isopropylamide, N-vinyl-N-ethyl formamide, and silicone-based monomers, oligomers, or macromers.

Functional monomers are known to have a double bond to interact with the ocular therapeutic agent. However, in some embodiments the functional monomers can have more than one double bond (i.e. vinyl oligomers or macromers) and can further function as crosslinking agents.

Exemplary silicone-based functional monomers include macromers silicone-based monomers or macromers including polysiloxane, polydimethyl siloxane, methacryloxypropyl terminated poly dimethylsiloxane (DMS-R11), tris(trimethylsiloxy)silyl propyl methacry late (TRIS): hydrophilic TRIS derivatives including tris(trimethylsiloxy)silyl propyl vinyl carbamate (TPVC), tris(trimethylsiloxy)silyl propyl glycerol methacry late (SIGMA), tris(trimethylsiloxy)silyl propyl methacry loxyethylcarbamate (TSMC), polydimethylsiloxane (PDMS): or monomers or macromers with pendent silicone groups including methacry late end-capped fluoro-grafted PDMS cross linker, a methacrylate end-capped urethane-siloxane copolymer cross linker, a styrene-capped siloxane polymer containing polyethylene oxide and polypropylene oxide blocks, siloxane containing hydrophilic grafts or amino acid residue grafts, and siloxanes containing hydrophilic blocks or containing amino acid residue grafts.

In some embodiments, the hydrogel matrix can comprise silicone-based functional monomers or other monomers in an amount of from about 50 wt-% to about 90 wt-%, wherein the silicone-based functional monomers or other monomers comprises silicone or siloxane oligomer or macromer (including Lotrafilcon A or B macromers, or Betacon macromers), macromers comprising two terminal methacryloxyethyl and/or methacryloxypropyl terminated groups and at least two polysiloxane or polydimethylsiloxane segments, methacryloxypropyl-tris-(trimethylsiloxy) silane (TRIS), and/or N,N dimethyl acrylamide (DMA).

In some embodiments, the hydrogel matrix can further comprise additional functional monomers comprising diethyl aminoethyl methacry late (DEAEM) and/or diallyl dimethyl ammonium chloride (DADMAC) in an amount of from about 0.01 wt-% to about 10 wt-%.

The cross-linked polymeric hydrogel matrix further comprise vinyl, bifunctional or multi-functional crosslinking agents, which can include low molecular weight, hydrophilic and hydrophobic crosslinking monomers and bi-functional crosslinking molecules (including those that are not monomers), and are referred to herein as “crosslinking agents”. Crosslinking agents can include molecules with reactive groups that can react with groups along the polymer chains (e.g. primary amines, sulfhydryls, carbonyls, carbohydrates, and carboxyls). As referred to herein the low molecular weight includes crosslinking agents that are less than about 1000 g/mol, preferably between about 1 and 800 g/mol. As one skilled in the art will ascertain from the disclosure herein, a plurality of crosslinking agents can be employed with the inclusion of at least one low molecular weight crosslinking agent. In some embodiments the crosslinking agents are lower molecular weight than the functional oligomers or macromers of the hydrogel matrix and having different hydrophilicity/hydrophobicity to provide controlled release in the low elastic modulus silicone hydrogel contact lenses. In embodiments, the hydrogel matrix comprises from about 0.5 wt-% to about 15 wt-% of the low molecular weight vinyl, bifunctional or multi-functional crosslinking agents.

Exemplary low molecular weight crosslinking agents include polyethylene glycol (200) dimethacrylate (PEG200DMA), ethylene glycol dimethacrylate (EGDMA), tetraethyleneglycol dimethacrylate (TEGDMA), N,N′-Methylene-bis-acrylamide, polyethylene glycol (600) dimethacrylate (PEG600DMA): 2,2-Bis[4-(2-hydroxy-3-methacryloxypropoxy)phenyl]propane, 1,10-Decanediol dimethacrylate; and the like.

In an exemplary embodiment, the hydrogel matrix can comprise a low molecular weight bi-functional crosslinking agent comprising ethylene glycol dimethacrylate (EGDMA) and/or polyethylene glycol 200 dimethacrylate (PEG200DMA) in an amount of from about 0.5 wt-% to about 15 wt-%.

In any of the embodiments the hydrogel matrix further comprises water, including in an amount between about 10 wt-% to about 50 wt-%.

In an exemplary embodiment, the silicone hydrogel contact lens delivery system comprises Lotrafilcon A or B macromers, methacryloxypropyl terminated poly dimethylsiloxane (DMS-R11), methacryloxypropyl-tris-(trimethylsiloxy) silane (TRIS), N,N dimethyl acrylamide (DMA), ethylene glycol dimethacrylate (EGDMA), polyethylene glycol (200) dimethacrylate (PEG200DMA), diethylaminoethyl methacry late (DEAEM), diallyl dimethyl ammonium chloride (DADMAC), methacrylamidopropyltrimethyl ammonium chloride (MAPTAC), bromfenac sodium, ethanol, 2-hydroxy-2-methylpropiophenone, or a combination thereof.

The silicone hydrogel contact lens of the delivery system has an elastic modulus of less than about 5.0 MPa, less than about 2.0 MPa, less than about 1.2 MPa, or less than about 1.0 MPa. In some preferred embodiments the silicone hydrogel contact lens has an elastic modulus between about 0.1 mPa and about 2.0 MPa, or between about 0.5 mPa and about 2.0 MPa. Beneficially and without being limited to a particular mechanism of action, the cross-linked polymeric hydrogel matrix provides a synergistic effect between memory site effectiveness and the elastic modulus that is related to the structural parameters of the network due in part to the use of varying sizes of crosslinking agents used to create the memory sites. This leads to macromolecular memory release control in contact lens delivery systems containing longer chain macromers that lower the elastic modulus of the silicone hydrogel contact lens.

In an embodiment, for low to moderate-sized drugs (e.g. 50-800 g/mol or 200-600 g/mol molecular weight), the crosslinking agents provide the delivery system with the extended drug release due to macromolecular memory.

Without being limited to a particular mechanism of action, the cross-linked hydrogel matrix made up of the vinyl, bifunctional (bi-vinyl) or multi-functional (multi-vinyl) monomers, oligomers, and/or macromers (i.e. functional monomers) along with the low molecular weight crosslinking agents copolymerize to form the memory sites. In particular, the inclusion of the variation in sizes of the monomers, oligomers, and macromers provides for the cross-linked polymeric hydrogel matrix as described herein, including the memory sites. For example, vinyl, bifunctional or multi-functional macromer composition of larger MW and higher concentration and lower molecular weight, vinyl, bifunctional or multi-functional crosslinking agent(s) of lower MW and lower concentration enhances the macromolecular memory creation for effective controlled release and allows a commercially acceptable elastic modulus of the hydrogel lens.

The silicone hydrogel contact lens of the delivery system has an oxygen permeability of at least about 50 Barrer, at least about 70 Barrer, at least about 90 Barrer, or at least about 110 Barrer.

The hydrogel matrix can further comprise a photo-initiator in an amount of from about 0) wt-% to about 5 wt-%. If a photo-initiator is included in the methods of making the silicone hydrogel contact lens of the delivery system, it is added in the formulation before the polymerization step. Exemplary photo-initiators include for example, 2-hydroxy-2-methylpropiophenone, azobisisobutyronitrile (AIBN), 2,2-dimethoxy-2-phenyl acetophenone (DMPA), 1-hydroxycyclohexyl phenyl ketone (Irgacure® 184), 2,2-dimethoxy-1,2-diphenylethan-1-one (Irgacure 651), ammonium persulfate, iniferter such as tetraethylthiuram disulfide, etc.

In any of the embodiments, the hydrogel matrix comprises a solvent in an amount of from about 0 wt-% to about 60 wt-%, or from about 20 wt-% to about 45 wt-%. A preferred solvent is water. Additional solvents including ethanol, dimethylsulfoxide (DMSO), isopropanol, etc. can be used in the methods of making the hydrogel matrix. In some embodiments a solvent is included in making the hydrogel matrix to aid with the biphasic compositions and provide optically clear lens with desired elastic modulus. However, in some embodiments no solvent is required.

The therapeutic ocular agent in the contact lens delivery system can comprise a drug, such as an antibiotic, an anti-inflammatory, an antihistamine, an antiviral agent, a cancer drug, an anesthetic, a cycloplegic, an anticholinergic, an antimuscarinic, a mydriatics, a lubricant agent, a hydrophilic agent, a decongestant, a vasoconstrictor, vasodilator, an immuno-suppressant, an immuno-modulating agent, an anti-glaucoma agent, an anti-infective, hyperosmolar agent, vitamins, growth factors, growth factor antagonists, sympathomimetics, an adrenergic agonist, an anti-cataract agent, an anti-hypertensive agent, an anti-macular degeneration agent, an ocular permeation enhancing agent, an anti-retinal disease agent, an anti-retinitis pigmentosa agent, an anti-diabetic retinopathy agent, an ocular myopia controlling agent, an ocular diagnostic agent, or combinations thereof. In embodiments one or more therapeutic ocular agents (i.e. drugs) is included in the contact lens delivery system.

In preferred embodiments, the drug is an anti-inflammatory agent comprising triamcinolone acetonide, dexamethasone, dexamethasone sodium phosphate, and other corticosteroids, bromfenac sodium, diclofenac sodium, and/or non-steroidal anti-inflammatory drugs (NSAIDs).

In further preferred embodiments, the drug is an antibiotic comprising moxifloxacin or other quinolone or fluoroquinolone antibiotics, cefuroxime and other cephalosporin antibiotics, vancomycin or other glycopeptide antibiotics (including teicoplanin, telavancin, ramoplanin and decaplanin, corbomycin, and complestatin), or combinations thereof.

In further embodiments, the hydrogel matrix contains from about 20 μg to about 500 μg, about 50 μg to about 250 μg, or about 80 μg to about 200 μg of the drug.

### Methods of Treatment

Methods of treating one or both eyes of a mammal in need thereof with a contact lens delivery system comprise contacting the contact lens delivery system to one or both eyes of the mammal to provide controlled release of the at least one ocular therapeutic agent to treat the eye(s) of the mammal. In preferred embodiments the mammal is a human. The embodiments of the contact lens delivery system as described herein are provided to contact the eye(s) of the mammal for such treatment.

In embodiments, the silicone hydrogel contact lens of the delivery systems can include afocal, multi-focal, vision correcting or non-correcting, plano, or bandage lenses having no vision correction.

The treatment methods can include the contact lens delivery system used for the treatment of a condition including post-cataract surgery, post-laser-assisted in situ keratomileusis (LASIK) or other forms of laser-assisted ocular and/or vision surgery, uveitis (acute, subacute, or chronic), and corneal abrasion. Examples of other forms of laser-assisted ocular and/or vision surgery can include photorefractive keratectomy (PRK) surgery, small incision lenticule extraction (SMILE) laser surgery, epithelial-LASIK surgery, lens replacement surgery or refractive lens exchange, laser cataract surgery, laser epithelial keratomileusis (LASEK) surgery, and PresbyLASIK or multifocal LASIK surgery.

In any of the embodiments, the therapeutic ocular agent in the contact lens delivery system can comprise a drug, such as an antibiotic, an anti-inflammatory, an antihistamine, an antiviral agent, a cancer drug, an anesthetic, a cycloplegic, an anticholinergic, an antimuscarinic, a mydriatics, a lubricant agent, a hydrophilic agent, a decongestant, a vasoconstrictor, vasodilator, an immuno-suppressant, an immuno-modulating agent, an anti-glaucoma agent, an anti-infective, hyperosmolar agent, vitamins, growth factors, growth factor antagonists, sympathomimetics, an adrenergic agonist, an anti-cataract agent, an anti-hypertensive agent, an anti-macular degeneration agent, an ocular permeation enhancing agent, an anti-retinal disease agent, an anti-retinitis pigmentosa agent, an anti-diabetic retinopathy agent, an ocular myopia controlling agent, an ocular diagnostic agent, or combinations thereof.

In exemplary embodiments, the drug is an anti-inflammatory agent comprising triamcinolone acetonide, dexamethasone, dexamethasone sodium phosphate, and other corticosteroids, bromfenac sodium, diclofenac sodium, steroidal anti-inflammatory drugs (SAIDs), and/or non-steroidal anti-inflammatory drugs (NSAIDs).

In further exemplary embodiments, the drug is an antibiotic comprising moxifloxacin or other quinolone or fluoroquinolone antibiotics, cefuroxime and other cephalosporin antibiotics, vancomycin or other glycopeptide antibiotics, including teicoplanin, telavancin, ramoplanin and decaplanin, corbomycin, and complestatin, or combinations thereof.

Exemplary treatment methods can include wherein the one or both eyes require treatment with any of the drugs described herein, for example steroidal anti-inflammatory drugs (SAIDs) and/or non-steroidal anti-inflammatory drugs (NSAIDs). Further exemplary treatment methods can include wherein the one or both eyes require treatment with any of the drugs described herein, for example antibiotics.

The treatment methods can include the silicone hydrogel contact lens contacting the one or both eyes continuously for a period of less than about 30 days. In preferred embodiments, the methods can include the silicone hydrogel contact lens contacting the one or both eyes continuously for a period of about 1 to about 30 days, a period of about 2 to about 20 days, a period of about 3 to about 15 days, a period of about 4 to about 10 days, or about 7 days. In addition, without being limited according to the disclosure, all ranges recited are inclusive of the numbers defining the range and include each integer within the defined range.

In embodiments, the treatment methods can include a duration of treatment that is for a period of between about 1 week to about 15 weeks, and wherein the silicone hydrogel contact lens are replaced every about 5 days to about 16 days, or every about 5 to about 10 days throughout the duration of treatment. In addition, without being limited according to the disclosure, all ranges recited are inclusive of the numbers defining the range and include each integer within the defined range.

In embodiments, the methods can include the effective amount of the at least one ocular therapeutic agent being increased or decreased when the silicone hydrogel contact lens are replaced during the duration of treatment. This beneficially provides the physician or treating medical provider to adjust the dosage of the ocular therapeutic agent(s) in the contact lens delivery system providing an unprecedented ability to provide patient-specific care with the contact lens delivery system.

Beneficially, the treatment methods with the contact lens delivery system is designed for the ocular therapeutic agent, i.e. drug, in the silicone hydrogel contact lens to have a release duration matching recall (i.e. follow-up) time of clinicians for patient in need of treatment thereof. For example, for cataract surgery, typical post-cataract surgery recall time is one week after surgery. Directly after surgery, clinicians will place the lens on the patient and lenses will be worn continuously for 7 days and be removed and replaced by the clinician on post-surgical 1 week check-up. The clinician will place the second lens on the patient to be worn continuously for another 7 days/1 week. Depending on the patient and their recovery, the clinician may set another 1 week return appointment for the patient to assess recovery and place the third lens to be worn for another week. Or the patient may change the lens after 14 days and place the third lens themselves or use a caregiver or staff at an office visit to place the lens. Treatment duration can vary from 2 to 6 weeks requiring 2 to 6 lenses worn with replacement every 7 days. Treatment may include a decreasing amount of drug delivered tapering from high to moderate to low or an increasing amount of drug delivered from low to high in the course of the 2 to 6 week treatment.

As a further example, for post-LASIK (laser-assisted in situ keratomileusis) and other forms of laser-assisted ocular or vision surgery such as PRK (photorefractive keratectomy) surgery, SMILE laser surgery, Epi-LASIK (epithelial-LASIK) surgery, lens replacement surgery or refractive lens exchange, laser cataract surgery, LASEK (laser epithelial keratomileusis) eye surgery, Presby LASIK or multifocal LASIK, etc., treatment will be typically 1 week post-surgery with lens placement by the clinician directly after surgery. Complications may require an additional week of treatment after follow-up with a second lens placement for a week. As a still further example, for uveitis, the clinical strategy is often to place a drug releasing lens after diagnosis with lens drug release duration matching recall/follow-up time of 1 week. If the patient does not show clinical signs of improvement, follow-up time is 1 week until improvement. Lens drug loading can be increased to deliver a more effective therapeutic concentration if poor clinical signs of improvement or flare. Once inflammation is reduced and has clinical signs of continual improvement, the follow-up examination schedule can be lengthened to every 2 weeks. Treatment may include a decreasing amount of drug delivered tapering from high to moderate to low or an increasing amount of drug delivered from low to high in the course of 6-10 week treatment. Lens drug loading can be increased or reduced in the lens to achieve an increase or decrease in drug ocular concentration for weekly (1 lens) or every 2 weeks/14 days (two 7-day wear lenses) treatment. As a still further example, for corneal abrasions, treatment will be typically 1 week post-surgery with lens placement by the clinician directly after surgery. With minor abrasions, healthy cells quickly fill the defect to prevent refraction irregularity and infection, and deeper abrasions can lead to corneal scarring with scarring leading to corneal transplant. With deeper abrasions, treatment may be an additional week after 1-week follow-up.

Based on the ability to tailor treatment methods with silicone hydrogel contact lens having an ocular therapeutic agent (i.e. drug) with a release duration matching recall, an exemplary treatment methods suitable for use of the contact lens delivery systems containing ocular therapeutic agent(s) include treating post-cataract surgery, wherein the duration of treatment is between about 1 week and about 8 weeks, and wherein the silicone hydrogel contact lens are replaced every about 5 to about 18 days throughout the duration of treatment, or every about 7 days.

Further exemplary treatment methods suitable for use of the contact lens delivery systems containing ocular therapeutic agent(s) include treating post-LASIK or other forms of laser-assisted vision surgery, wherein the duration of treatment is between about 5 days and 3 weeks, and wherein the silicone hydrogel contact lens are replaced every about 5 to about 10 days throughout the duration of treatment, or every about 7 days.

Further exemplary treatment methods suitable for use of the contact lens delivery systems containing ocular therapeutic agent(s) include treating uveitis, wherein the duration of treatment is between about 4 weeks to about 12 weeks, and wherein the silicone hydrogel contact lens are replaced every about 5 to about 10 days throughout the duration of treatment, or every about 7 days.

Still further exemplary treatment methods suitable for use of the contact lens delivery systems containing ocular therapeutic agent(s) include treating corneal abrasion, wherein the duration of treatment is between about 5 days and about 3 weeks, and wherein the silicone hydrogel contact lens are replaced every about 5 to about 10 days throughout the duration of treatment, or every about 7 days.

In any of the embodiments, beneficially the bioavailability of the contact lens delivery system exceeds that of a conventional eye drop therapy for the same indication of use, and wherein preferably the bioavailability of the contact lens delivery system is at least 5 times greater, at least 10 times greater, at least 15 times greater, or at least 20 times greater than a conventional eye drop therapy for the same indication of use and comparing the same tissue or tear film values.

In any of the embodiments, the contact lens delivery system provides a bioavailability in tear film (AUC0-8 days) of at least about 140 μg week/mL, or at least about 1,000 μg week/mL when provided at a Cₘₐₓ concentration of at least about 200 μg/mL. In any of the embodiments, the contact lens delivery system provides a bioavailability in tear film (AUC₀₋₂₄ hours) of at least about 20 μg day/mL, or at least about 100 μg day/mL when provided at a Cₘₐₓ concentration of at least about 200 μg/mL.

In any of the embodiments, the contact lens delivery system provides an average concentration to the eye of the at least one ocular therapeutic agent of between about 0.005 μg/mL to about 400 μg/mL per day, about 0.01 μg/mL to about 400 μg/mL per day, about 0.1 μg/mL to about 400 μg/mL per day, about 1 μg/mL to about 400 μg/mL per day, or 2.5 μg/mL to about 400 μg/mL per day based on tissue density. In embodiments, the contact lens delivery system provides an average concentration to the eye of the at least one ocular therapeutic agent of at least about 0.005 μg/mL per day, at least about 0.01 μg/mL per day, at least about 0.1 μg/mL per day, at least about 1 μg/mL per day, at least about 10 μg/mL per day, or at least about 100 μg/mL per day based on concentration or tissue density.

Moreover, in any of the embodiments, the contact lens delivery system provides an average ocular tissue (e.g., cornea, sclera, choroid, iris/ciliary body, etc.) or aqueous humor concentration of at least about 0.005 μg/mL per day, at least about 0.01 μg/mL per day, at least about 0.1 μg/mL per day, at least about 1 μg/mL per day, at least about 10 μg/mL per day, or at least about 100 μg/mL per day.

In any of the embodiments, the methods can further include the one or both eyes further treated with an antibiotic with an optional long-acting SAID via intracameral irrigation or injection, subconjunctival or sub-Tenon's injection, or intravitreal injection and/or depot placement prior to the contacting of the contact lens delivery system to the one or both eyes of the mammal.

### EXAMPLES

Embodiments of the present embodiments of the disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Maintaining all lens properties within commercial design tolerances as well as providing a mechanism for well-controlled drug release has proved to be a major challenge. Numerous methods have been studied to control the release of therapeutics from hydrogel contact lenses (including silicone hydrogel contact lenses) including equilibrium partitioning, diffusion barriers, carrier-mediated release, inclusion complexes, molecular imprinting, and others. A thorough analysis of in vivo studies highlight that all systems either exhibit poor drug delivery control (i.e., majority of drug payload released very quickly, little control over release rate and drug concentration with release kinetics not matching duration of wear, failure to deliver a therapeutically relevant drug concentration) and/or lack critical lens physical properties needed to commercialize (oxygen transport, clarity, elastic modulus, water content, etc.).

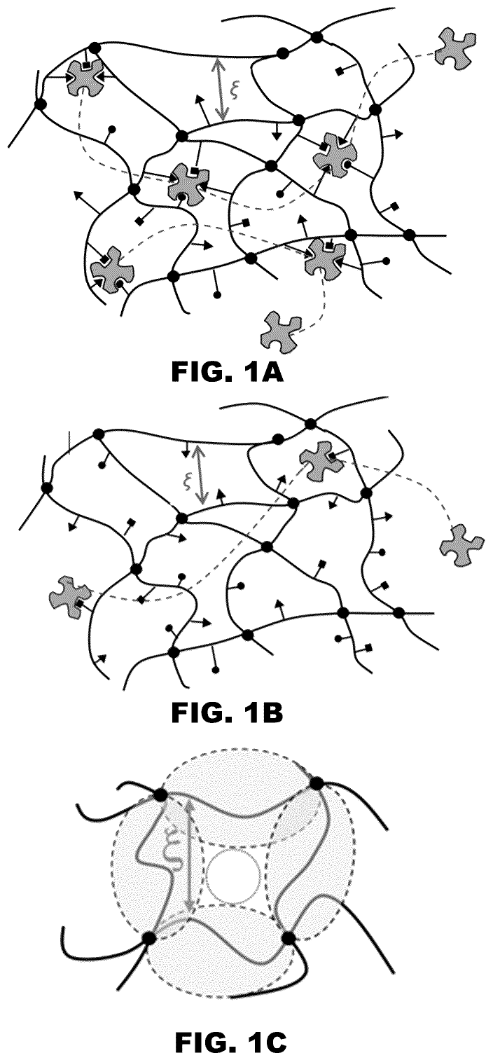

The contact lens delivery systems described herein provide extended-wear silicone hydrogel contact lens that meet all commercial lens property standards with controlled release of an ocular therapeutic agent, such as a non-steroidal anti-inflammatory drug (NSAID), with an in vivo therapeutic concentration for extended, continuous duration of wear, such as over seven days or greater. Utilizing a macromolecular memory strategy where a macromolecular framework for the ocular therapeutic agent is produced during lens synthesis, strict control over the ocular therapeutic agent loading and release is herein described and generally depicted in FIG. 1. Memory sites or macromolecular memory are formed for loading of an ocular therapeutic agent (FIG. 1A). The memory sites are made by the templating process during polymerization leading to non-covalent complexation points on multiple polymer chains arranged within the network. Drug loading and release duration are increased over control lens (FIG. 1B) which are polymerized without drug leading to randomly incorporated chemical functionality. The average mesh size (%) of the gel (arrow) has a synergistic effect on drug loading and release (FIG. 1C) and the polymers have mobility leading to excluded volume. Dotted lines in FIG. 1A and FIG. 1B indicate diffusion path within and out of lens.

### Example 2

### Synthesis of Silicone Hydrogel Contact Lenses

Methacryloxypropyl terminated polydimethylsiloxane (DMS-R11) and methacryloxypropyl-tris-(trimethylsiloxy) silane (TRIS) were purchased from Gelest, Inc. (Morrisville, PA). N,N dimethyl acrylamide (DMA), ethylene glycol dimethacrylate (EGDMA), polyethylene glycol (200) dimethacrylate (PEG200DMA), diethyl aminoethyl methacrylate (DEAEM), diallyl dimethyl ammonium chloride (DADMAC), acrylic acid (AA), methacrylic acid (MAA), dexamethasone sodium phosphate (DMSP), diclofenac sodium (DS), bromfenac sodium (BS), and moxifloxacin (MOX), ethanol, and 2-hydroxy-2-methylpropiophenone were purchased from VWR (Radnor, PA).

Silicone hydrogel contact lenses were synthesized using various mixtures of DMS-R11, TRIS, and DMA in addition to PEG200DMA, EGDMA, DEAEM, AA, MAA, DADMAC, and ethanol with MOX, BS, DMSP, or DS added to the prepolymer formulation (i.e. solution before polymerization) in various combinations. Photo-initiator 2-hydroxy-2-methylpropiophenone, was added at a composition of <1% of total formulation.

Functional monomers were added at various monomer to template (M/T) ratios for each drug equating to up to 10 mol % of total formulation. M/T ratio refers to the molar ratio of the functional monomer to the template drug and dictates the amount of drug added to the prepolymer formulation such that no more than 10 mol % of the total formulation is functional monomer. Functional monomers were selected based on their ability to non-covalently complex with drug molecules. DEAEM and DADMAC were selected due to their positive charge and ability to form ionic bonds with negatively charged template molecules while MAA and AA were chosen to form hydrogen bonds with templates molecules that did not possess a charge. M/T ratios were normalized to the highest M/T ratio amongst all formulations. Control lenses were synthesized using the same macromers and monomers but without addition of template drug to the pre-polymer formulation. The pre-polymer formulation was vortexed for approximately 1 minute and then sonicated for 30 minutes at room temperature to remove dissolved gases and ensure full dissolution of the template drug.

A volume of 65 μL of the pre-polymer formulation was pipetted into polypropylene lens molds (dimensions swollen silicone lens 14.8 mm diameter, 8.4 base curve). Polymerization occurred via UV polymerization using an Omnicure S2000 (Excelitas Technologies Corp., Waltham, MA), with an intensity of approximately 40 mW/cm² for a duration of 2 minutes. UV effects on the chemistry of loaded drugs was verified via 1H-NMR (400 MHZ, Agilent Technologies, Santa Clara, CA) to ensure that UV polymerization did not affect the chemical structure. Mass of drug within the lens was determined via drug uptake and release experiments via mass balance.

### Template Drug Binding Studies

All lenses were washed in 700 mL to 1 L of PBS in a Sotax AT Xtend Dissolution System (Sotax, Westborough, MA) at 30 rpm. To verify washing, lenses were removed and placed in 2 mL of PBS and supernatant drug concentration was measured until no drug was observed releasing from the lens (lower limit of detection of ˜0.5 μg/mL). Lenses that displayed additional drug elution were placed back in the dissolution apparatus. Effectiveness of the wash was determined via mass balance analysis during washing and release based on the mass of drug loaded within the lens, with over 95% of the loaded drug released during the washing process. Template binding studies were performed by placing washed lenses of different M/T ratios in 3 mL of 150 μg/mL drug solution (BS, DS, or DMSP, in DI water) until equilibrium was reached, which was verified experimentally. Equilibrium concentration of the supernatant was measured via UV/Vis spectrophotometry (280 nM) and used to determine mass uptake via mass balance. For dry lens mass, lenses were dried in a vacuum oven (T=30° ° C., 28 in. Hg vacuum) until weight change was less than 0.1% and dry masses were measured. Normalized drug mass uptake (μg drug/mg polymer) was determined for each M/T ratio. Control lenses were synthesized, washed, and loaded via the same method as templated lenses and analyzed for drug mass uptake. Imprinting factor for lenses at each M/T ratio was calculated by dividing normalized drug mass uptake by normalized drug uptake observed in controls.

Drug molecules added within the prepolymer formulation complex with functional monomers, beginning the templating process. During polymerization, these complexes create complexation points within multiple polymer chains which form macromolecular memory sites within the polymer structure. Drug reloading, dynamic release experiments, and network structural analysis have been validated with various drugs.

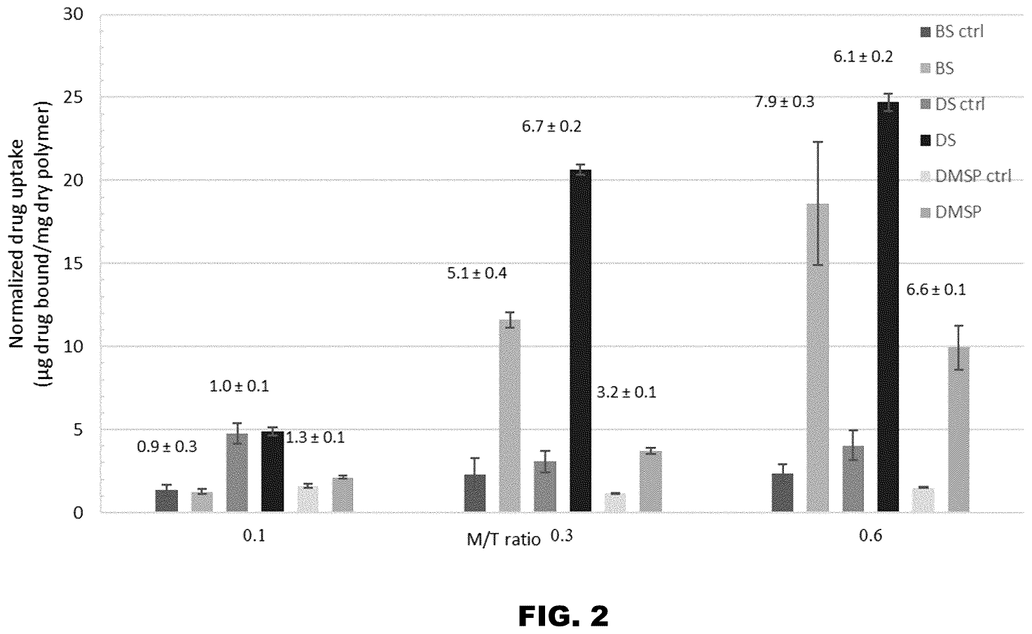

Equilibrium mass binding of DMSP, DS, and BS in templated silicone hydrogel contact lenses at different M/T ratios and controls are shown in FIG. 2. DMSP templated lenses demonstrated equilibrium binding values of 2.1±0.1 μg_{drug}/mg_{polymer}, 3.7±0.2 μg_{drug}/mg_{polymer}, and 9.9 #1.3 μg_{drug}/mg_{polymer} corresponding with normalized M/T ratios of 0.1, 0.3, and 0.6 respectively. Imprinting factor for DMSP templated lenses synthesized at M/T ratios of 0.1, 0.3, and 0.6 were 1.3±0.1, 3.2±0.1, and 6.6±0.1 respectively, demonstrating an increase in drug binding compared to controls (imprinting factor above 1 compared to control) and demonstrating that macromolecular memory sites within the lens lead to an increase in drug uptake. DS templated lenses at different M/T ratios demonstrated equilibrium binding values of 4.9±0.3 μg_{drug}/mg_{polymer}, 20.6±0.3 μg_{drug}/mg_{polymer}, and 24.7±0.5 μg_{drug}/mg_{polymer} corresponding with normalized M/T ratios of 0.1, 0.3, and 0.6 respectively and imprinting factors of 1.0±0.1, 6.7±0.2, and 6.1±0.2 respectively. Equilibrium mass binding of BS in BS templated lenses with M/T ratios of 0.1, 0.3, and 0.6 were 1.3±0.2 μg_{drug}/μg_{polymer}, 11.6±0.5 μg_{drug}/μg_{polymer}, and 18.6±3.7 μg_{drug}/μg_{polymer} respectively, corresponding with imprinting factors of 0.9±0.3, 5.1±0.4, and 7.9=0.3 respectively.

Equilibrium binding results for DS, DMSP, and BS demonstrated an increased drug uptake as M/T ratio increased. Controls demonstrated the lowest drug binding while the highest M/T ratios demonstrated the highest drug binding, with higher M/T ratios binding significantly more mass than controls synthesized with the same mol % of functional monomer. These results show that macromolecular memory sites lead to a higher drug uptake and increasing functionality within the lens leads to a higher degree of macromolecular memory site formation in lenses loaded via the templating process as the template drug. Controls in this study contained functionality that matched the template drug at the same concentration as templated lenes with the only difference being the absence of template drug in the prepolymer formulation in controls. This example demonstrates that the templating process leads to macromolecular memory site formation which enhances drug uptake rather than only the presence of functional chemistry that interacts with the template drug.

### In Vitro Physiological Flow Release

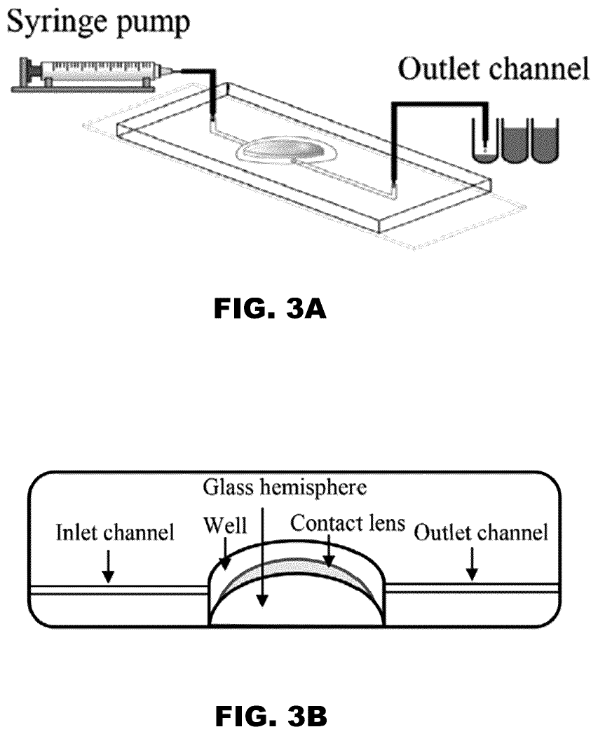

Release studies were conducted via an in vitro physiological flow model using a microfluidic device as shown in FIGS. 3A-3B. The device was produced using poly dimethylsiloxane (PDMS). A mixture of 10:1 ratio of Sylgard 184 Silicone base and curing agents was prepared and stirred for 4 minutes, then poured onto a glass plate within a circular mold. Two needles (1.27 mm outer diameter) were placed into the mold to create an inlet and outlet for flow, and a hemisphere on the glass plate (9.00±0.10 mm radius of curvature) created a well in which contact lenses were placed during release. The device was then cured at 60° C. for 6 hours. Drug loaded lenses were placed in the well of the device and fixed into position with a glass hemisphere (8.75±0.10 mm radius of curvature). The microfluidic device was then sealed onto a glass plate using metal clamps. A kd Scientific Model 220 syringe pump (kd Scientific Inc., Holliston, MA) was used to inject solution (DI water or PBS) at ambient temperature (25° C.) through the device at a rate of 3 μL/min, simulating physiological tear flow. Prior to release analysis, lenses were fully washed until no additional drug was observed eluting from the lens and then reloaded with the template drugs. Release samples were collected and analyzed at different time intervals via HPLC (Waters Corp, Milford, MA) with tandem UV/Vis detector at a wavelength of 280. HPLC conditions consisted of a C18 column (3.8 μm diameter, Waters Corp, Milford, MA) and mobile phase of 50% acetonitrile and 50% aqueous (1% formic acid, v/v).

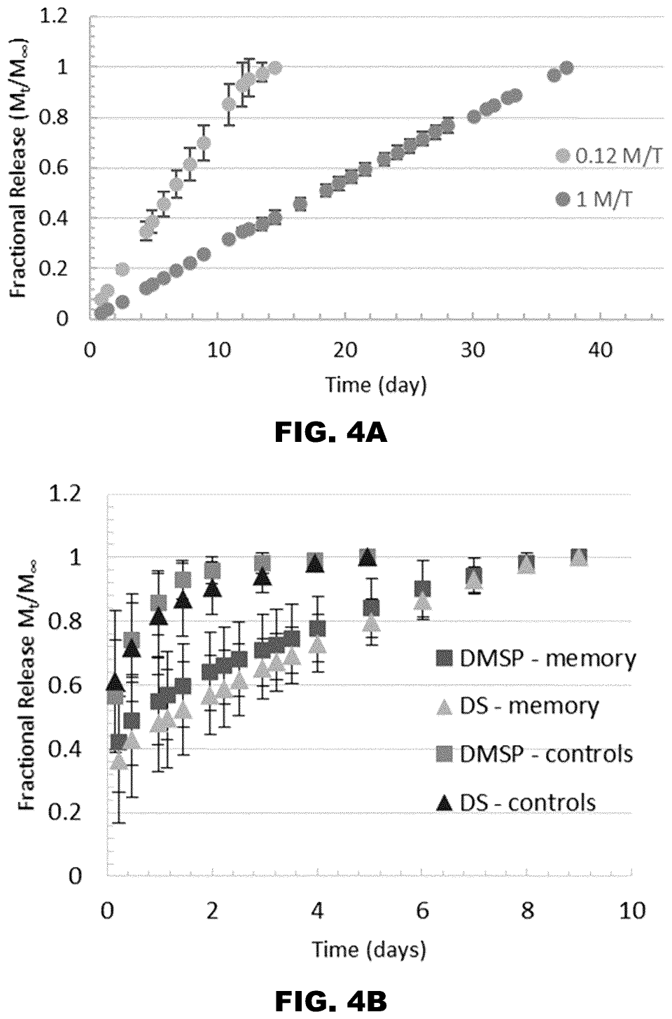

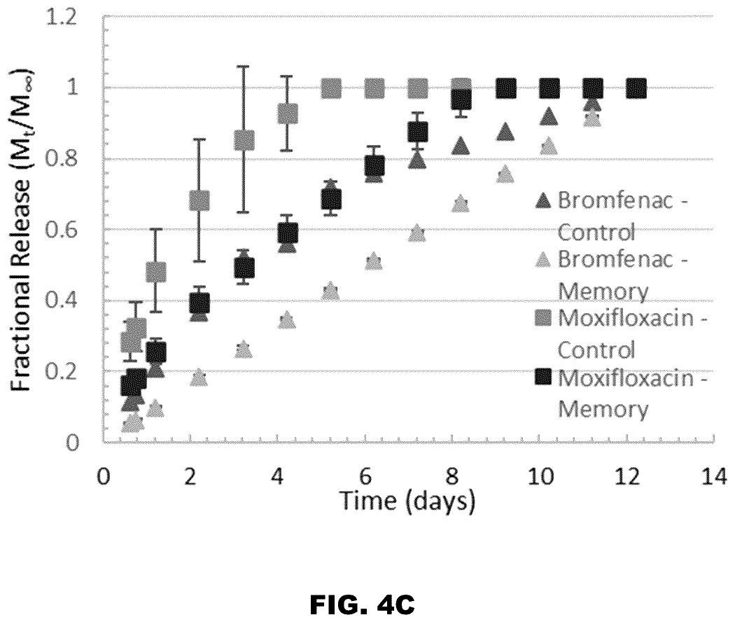

Release via the microfluidic physiological flow device has been demonstrated by the Applicant and inventors to be a more effective method for correlation of in vitro results to in vivo. Release via the microfluidic device more accurately replicates volume and flow dynamics within the tear film to more accurately predict in vivo drug release behavior of drug loaded lenses. Release results of BS loaded templated lenses synthesized at normalized M/T ratios of 1.0 and 0.12 are demonstrated in FIG. 4A. Lenses synthesized at an M/T ratio of 0.12 released their drug payload in 14 days while lenses synthesized at an M/T ratio of 1.0 extended release up to 35 days, showing that an increase in functionality within the lens leads to an increase in memory site formation during synthesis, resulting in a decreased release rate. Average mass released from lenses synthesized with an M/T ratio of 0.12 was 4.6±0.2 μg/day, while average mass release from 1.0 M/T lenses was 4.4±0.1 μg/day.

FIG. 4B shows in vitro microfluidic fractional dual release of DS and DMSP from DS+DMSP templated lenses and controls. Release of both DS and DMSP from control lenses occurred rapidly, with approximately 85% of the drug payload within the first day. By the second day, over 95% of loaded DMSP was released, with the remaining small amount of drug (<5%) released by the following day. Approximately 90% of loaded DS had been released by day 2 with the remaining 10% released over the following 2 days. Drug release profiles from controls are shown to be slightly better than soaking commercial lenses, as controls contain functional monomers that non-covalently interact with the template drug but lack hypothesized polymer chain templating organization formed in presence of drug. Lenses synthesized with the templating process extended release of both DS and DMSP to over 7 days and shifted the release curve downwards towards a more constant release rate. Dual release of BS and MOX from lenses synthesized with the templating process and controls are shown in FIG. 4C. Lenses synthesized using the templating process showed MOX release for 8 days and BS release for 11 days. Controls demonstrated a faster release of MOX, with ˜40% of the payload released within the first day and the majority released before day 4. Controls demonstrated 11 day release of BS, at a rate shifted to the left of templated lenses signifying a release profile that is less controlled and concentration dependent (further from zero order controlled release). Templated DMSP+DS loaded lenses released DMSP and DS at an average rate of 6.8±1.9 μg/day and 11.4±2.8 μg/day respectively while templated BS+MOX loaded lenses released BS at an average rate of 28.2±8.6 μg/day and MOX at an average rate of 14.0±5.0 μg/day. DMSP topical drops (0.1%, Maxidex) are administered 4-6 times daily and DS topical drops (0.1%, Voltaren) are administered 4 times daily. Assuming a drop volume of 50 μL, each drop delivers approximately 50 μg of medication, resulting in 200 μg/day of applied DS and 200 μg/day of applied DMSP (4 drops/day). For topical drops, approximately 92% of the applied therapeutic is lost due to tear turnover, resulting in an estimated therapeutic dosage of 16 μg/day of both DS and DMSP. Moxifloxacin topical drops (0.5%, Vigamox) are administered once daily, resulting in 500 μg/day of applied moxifloxacin and an estimated 40 μg/day dosage taking tear turnover into account. Bromfenac topical drops (0.09%, Xibrom) are administered twice daily, resulting in 90 μg/day of applied bromfenac and an estimated 7.2 μg/day dosage considering tear turnover. Release rates from therapeutic lenses approximates the expected therapeutic dosage of topical drops, however via alteration of the M/T ratio, the release rate can be tailored to achieve a different dosage. Furthermore, it has been demonstrated that with a controlled release strategy, where lens release rate approaches absorption rate into tissue, losses of drug due to tear turnover are substantially reduced.

Results from drug reloading and release analysis show synthesizing lenses in presence of drug molecules and monomers with functional chemistry with affinity for the template drug resulted in an increase in drug binding and a slower, more controlled release. These results show the templating process forms macromolecular memory sites within synthesized lenses that delayed release and increased drug binding compared to controls. Results from BS release at different M/T ratios show that increasing functionality within the lens leads to a greater degree of memory site formation which led to an increased release duration. ¹H-NMR analysis demonstrated no difference in chemical structure between template drugs that had been subjected to UV polymerization and release from therapeutic lenses and drugs measured without any modification.

### Physical Property and Structural Analysis

To determine optical transmittance, transmittance of visible light (450-700 nm) was measured through circular hydrogel lens segments, cut with a cork borer with a diameter of 1.5 mm. Each lens segment was placed in the bottom of a 96 well plate and hydrated in 200 μL of DI water along with a blank well containing only 200 μL of water, with care taken to ensure that there were no air bubbles present in any wells. Absorbance values of each well was measured in a Tecan Infinite M200 Pro spectrophotometer (Tecan, Männedorf, Switzerland) and absorbance values of blank wells were subtracted from wells containing lenses.

Contact angle with water was measured via sessile drop contract angle goniometry (Theta Flex Tensiometer, Nanoscience Instruments, Phoenix, AZ). Contact lenses were plasma coated in a SPI Plasma Prep III Plasma Cleaner (SPI supplies, West Chester PA), and 5 mm circular cutouts were cut from the lenses with a cork borer. Using a micropipette, a water droplet was placed on the surface of cutouts and contact angle was measured.

Elastic modulus was measured via synthesis of rectangular drug eluting silicone hydrogel sheets via UV photopolymerization using glass slides separated by 500 μm Teflon spacers. Dumbbell shaped tensile testing strips were cut from these sheets and analyzed for elastic modulus using a Shimadzu EZ-SX tensile tester (Shimadzu, Kyoto, Japan) at a gauge length of approximately 18 mm and stretched at a rate of 5 mm/min. Elastic modulus was determined by measuring the initial slope of the stress/strain curve. Hydrogels remained hydrated for the duration of the tests via aerosol diffusion of water.

Edge-corrected Dk was calculated according to ISO 18369.4 (Ophthalmic Optics—Contact Lenses—Part 4: Physiochemical Properties of Contact Lens Materials). Lenses swollen in PBS were stacked to create polymers of different center thicknesses, measured using an electronic micrometer. Each lens or lens stack was placed on a polarographic oxygen sensor (Createch/Rehder Dev Co., Greenville, SC) with 8.7 mm base curve and analyzed using a 201T oxygen permeameter.

Equilibrium weight swelling ratio was determined by measuring the ratio of the swollen polymer weight to the dry weight. Synthesized lenses were dried until weight change was less than 0.1% in a vacuum oven and weight of the dried lenses was recorded. Lenses were swollen in DI water and swollen mass was recorded. Equilibrium weight swelling ratio was calculated using the relationship:

$$q = \frac{{W}_{S} - {W}_{d}}{{W}_{d}}$$

where q is equilibrium weight swelling ratio, Wₛ is weight of the swollen gel, and W_{d} is weight of the dry gel.

Equilibrium volume swelling ratio was determined by measuring the ratio of the swollen volume to the dry volume. Volume of dried and swollen gels were determined using Archimedes principle. Equilibrium volume swelling ratio was determined using the relationship:

$$Q = \frac{1}{{υ}_{S}} = \frac{V s}{{V}_{d}}$$

where Q is equilibrium volume swelling ratio, vₛ is polymer volume fraction in the swollen state, Vₛ is the volume of the swollen gel at equilibrium, and V_{d} is the volume of the dry gel.

The average molecular weight between crosslinks was calculated by analyzing tensile properties of synthesized polymers as well as polymer volume fractions. The relationship to calculate molecular weight between crosslinks was as follows:

$$E = ( \frac{RT {υ}_{2 , s}^{1 / 3}}{\overline{υ} {\overline{M}}_{c}} ) * ( 1 - \frac{2 {\overline{M}}_{c}}{{M}_{n}} )$$

where E is the tensile modulus, R is the ideal gas constant, T is temperature, Mₙ is the number average molecular weight of the polymer chains, v_{2,s} is the polymer volume fraction in the swollen state, v is the specific volume of the swollen polymer, and Me is the average molecular weight between crosslinks.

Average molecular weight between crosslinks was used to calculate the average mesh size of synthesized polymers using the relationship:

$$ξ = {{υ}_{2 , s}^{- 1 / 3} ( \frac{2 {C}_{n} {\overline{M}}_{c}}{{M}_{r}} )}^{1 / 2} l ,$$

where ξ is mesh size, v_{2,s} is the polymer volume fraction in the swollen state, Mᵣ is molecular weight of the repeat unit, M_{c} is the average molecular weight between crosslinks, Cₙ is the Flory characteristic ratio, and l is the length of the bond along the polymer backbone. Average molecular weight between crosslinks and mesh size were normalized to the highest values for each amongst all formulations.

Measured physical properties of DS+DMSP loaded lenses and BS+MOX loaded lenses are presented in Tables 1A and 1B. All tests were performed with at least three replicates. Elastic modulus of DMSP+DS loaded lenses was 3.4±0.6 MPa. Elastic modulus of BS+MOX loaded lenses was 2.1±0.5 MPa.

TABLE 1A

| | |
|---|---|
| Elastic modulus (MPa) | 3.4 ± 0.6 |
| Contact angle | 16.4° ± 3.1° |
| Equilibrium weight swelling ratio | 0.30 ± 0.09 |
| Polymer volume fraction | 0.86 ± 0.01 |
| Dk (barrer) | 83 (95% CL: 70-101) |
| Dimension swollen lens BC | 8.4/14.8 |
| (mm)/Dia (mm) | |
| (lens mold swelling/expansion ~5%) | |
| Center Thickness (mm) | ~0.085 |
| Duration of wear | 6 nights & 7 days/1 week |

TABLE 1B

| | |
|---|---|
| Elastic modulus (MPa) | 2.1 ± 0.5 |
| Contact angle | 22.6° ± 1.2° |
| Equilibrium weight swelling ratio | 0.20 ± 0.03 |
| Polymer volume fraction | 0.86 ± 0.02 |
| Dk (barrer) | 70 (95% CL: 53-103) |
| Dimension swollen lens BC | 8.4/14.8 |
| (mm)/Dia (mm) | |
| (lens mold swelling/expansion ~5%) | |
| Center Thickness (mm) | ~0.085 |
| Duration of wear | 6 nights & 7 days/1 week |

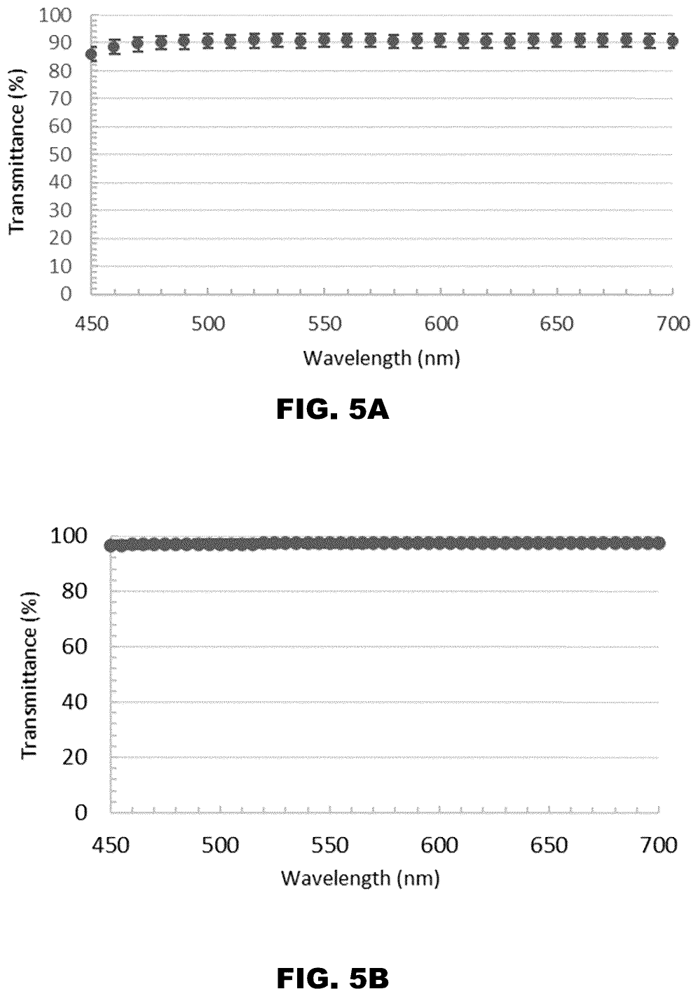

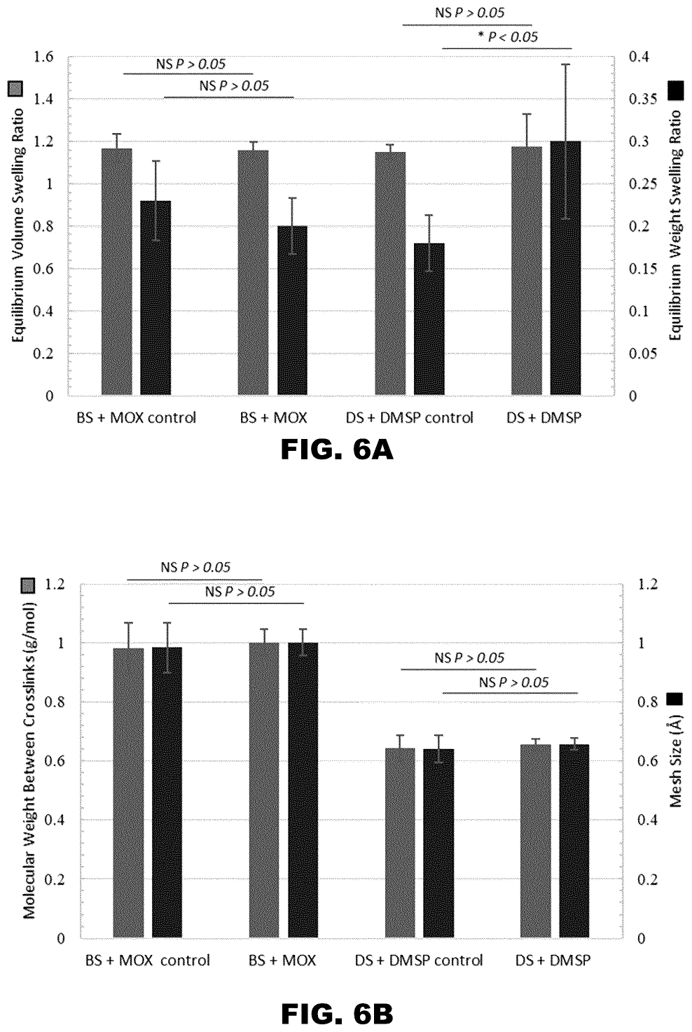

Elastic modulus of silicone hydrogel contact lenses generally ranges from 0.3-1.9 MPa, and is a tailorable property that can be adjusted by adjusting the amount of base monomeric units, using a longer chain silicone macromer unit, or using longer crosslinking units that allow for a more flexible polymer network. Contact angle of with water of DS+DMSO loaded lenses was determined to be 16.4°±3.1°, meeting the commercial standard for contact lenses of <100°. BS+MOX loaded lenses also met this commercial standard, displaying a contact angle with water of be 22.6°±1.2°. Oxygen permeability (Dk) analysis resulted in a Dk of 83 barrer (95% CL: 70-101) or 83×10⁻¹¹ (cm²/sec)(ml O₂/ml×mm Hg) at 35° C. (Dk intrinsic) in DS+DMSO loaded lenses and 70 barrer (95% CL: 53-103) at 35° C. in BS+MOX loaded lenses. These values fall within the range of extended-wear silicone hydrogel lenses on the market today (60-175). Light transmittance through DS+DMSP loaded lenses and BS+MOX loaded lenses was ≥96% @ 610 nm and greater than 90% across the visible spectrum, indicating that all lenses were optically clear (FIGS. 5A-5B). Equilibrium weight swelling ratios of lenses loaded with DS+DMSP was 0.29±0.09 compared to 0.18±0.03 in controls and 0.20±0.03 in templated lenses loaded with BS+MOX compared to 0.23±0.05 in controls (FIG. 6A-6B), fitting within the acceptable range for silicone hydrogel contact lenses.

Polymer volume fraction in the swollen state of DS+DMSP templated lenses was 0.86±0.03 compared to 0.86±0.05 in controls and 0.86±0.02 in BS+MOX templated lenses compared to 0.87±0.03 in controls. Normalized average molecular weight between crosslinks and mesh size of DS+DMSP templated lenses at an M/T ratio of 0.2 and corresponding controls as well as BS+MOX templated lenses at an M/T ratio of 0.2 and corresponding controls are highlighted in FIGS. 6A-6B. Structural analysis indicated that for both BS+MOX templated lenses and DS+DMSP templated lenses, lenses synthesized with the templating process had a mesh size that was not statistically different than controls. These results demonstrate that formation of macromolecular memory sites lead to extended release and increased drug loading in templated lenses rather than a tighter polymer architecture or smaller mesh size.

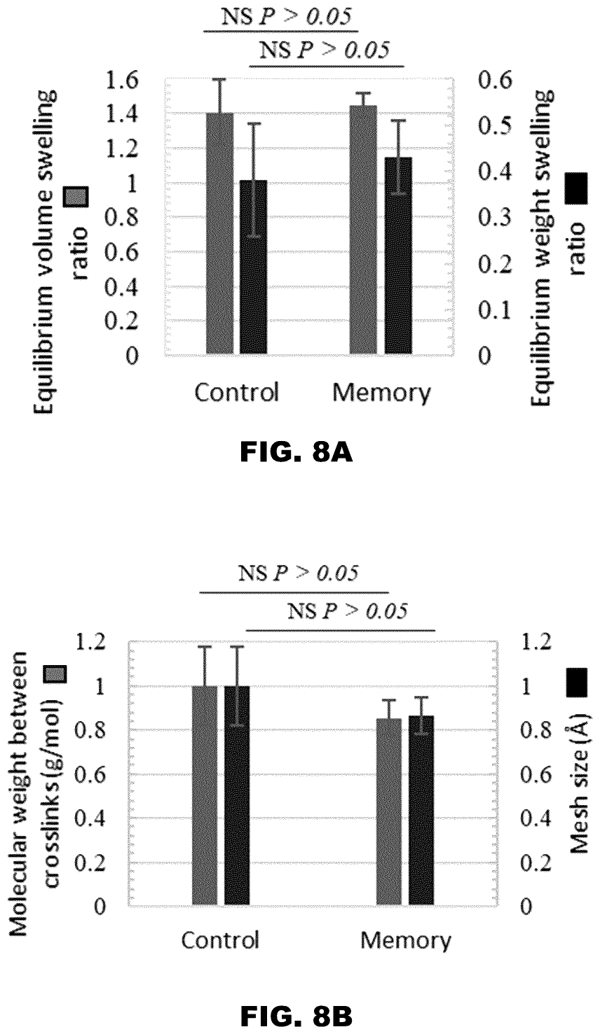

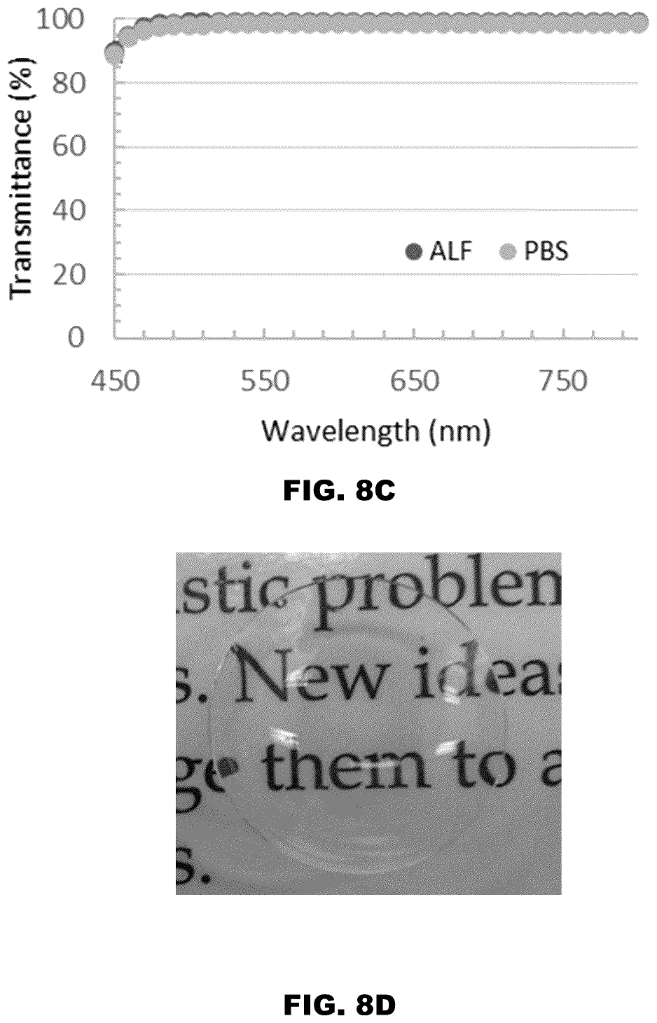

Measured physical properties of Bromfenac Extended-Release Contact Lenses (BERCLs) synthesized with macromolecular memory for bromfenac sodium are tabulated in Table 2 and are within specifications of commercially successful, extended-wear silicone hydrogel lenses on the market today. The physical properties of BERCLs synthesized with macromolecular memory for bromfenac sodium are shown in Table 2. All analyses were performed with three to six replicates, errors bars represent mean±SD. The elastic modulus of BERCLs was 0.6±0.3 MPa, matching commercial ranges for silicone hydrogel contact lenses, which range from 0.4-1.4 MPa. Measured water content of BERCLs was 43%±7%, at ambient temperature (23±2° C.), fitting within acceptable ranges of silicone hydrogel lenses on the market today. Oxygen permeability (Dk) analysis resulted in a Dk of 91.7 barrer or 91.7×10⁻¹¹ cm²/sec)(ml O₂/ml×mm Hg) at 35° C. (Dk intrinsic), which is also within the range of extended-wear silicone hydrogel lenses on the market today (60-175). The light transmittance of BERCLs were ≥96% @ 610 nm and greater than 90% across the visible spectrum, indicating that the BERCLs are optically clear (FIG. 8C). Refractive index and swollen lens dimensions such as base curve, diameter, center thickness, and swelling/expansion are within acceptable ranges of lenses on the market today, and these properties did not change with drug release (drug is <1% of lens formulation).

Lotrafilcon B contact lenses are one of the most successful contact lenses on the market and were FDA approved under PMA P010019 S003 on Sep. 27, 2004 for the optical correction of refractive ametropia (myopia and hyperopia) in phakic or aphakic persons with non-diseased eyes for up to 6 nights of extended-wear. The water content of lotrafilcon B lenses is specified as 33% at ambient temperature (23±2° C.), the oxygen permeability is 110×10⁻¹¹ cm²/sec)(ml O₂/ml×mm Hg) at 35° C. (Dk intrinsic), the refractive index (hydrated): 1.42 and Light Transmittance: ≥96% (@ 610 nm, −1.00D).

TABLE 2

| Physical Property | Value |
|---|---|
| Elastic modulus (MPa) | 0.6 ± 0.3 |
| Water uptake (%) | 43% ± 7% |
| Contact angle | 21.4° ± 0.85° |
| Dk (barrer) (ALF) | 91.7 (95% CL: 70.5-131.8) |
| Optical clarity(@ 610 nm/visible | ≥96%/>90% |
| spectrum) | |
| Polymer volume fraction swollen | 0.69 ± 0.03 |
| Specific gravity (ALF)/(PBS) | 1.03 ± 0.02/1.03 ± 0.09 |
| Refractive index (ALF)/(PBS) | 1.34 ± 0.06/1.34 ± 0.06 |
| Dimension swollen lens BC | 8.4/14.8 |
| (mm)/Dia (mm) | |
| (lens mold swelling/expansion ~5%) | |
| Center Thickness (mm) | ~0.085 |
| Duration of wear | 6 nights & 7 days/1 week |

### Example 3

### Synthesis of Silicone Hydrogel Contact Lenses

Methacryloxypropyl terminated polydimethylsiloxane (DMS-R11) and methacryloxypropyl-tris-(trimethylsiloxy) silane (TRIS) were purchased from Gelest, Inc. (Morrisville, PA). N,N dimethyl acrylamide (DMA), ethylene glycol dimethacrylate (EGDMA), polyethylene glycol (200) dimethacrylate (PEG200DMA), diethylaminoethyl methacrylate (DEAEM), diallyl dimethyl ammonium chloride (DADMAC), methacrylamidopropyltrimethyl ammonium chloride (MAPTAC), bromfenac sodium, ethanol, hexane, and 2-hydroxy-2-methylpropiophenone were purchased from VWR (Radnor, PA). Lotrafilcon B (LFB) formulation was provided by Gelest, Inc. (Morrisville, PA). All chemicals were used as received.

Various bromfenac extended-release contact lenses (BERCLs) were prepared using various mixtures of DMS-R11, TRIS, DMA, and Lotrafilcon B (LFB) silicone macromer formulation (90-95 mol % of total formulation) in addition to EGDMA, PEG200DMA, DEAEM, MAPTAC, DADMAC, and ethanol. Photo-initiator, 2-hydroxy-2-methylpropiophenone, was added at a composition of <1% of total formulation. Bromfenac sodium was dissolved in the pre-polymer formulation. Monomers were added at various monomer to template bromfenac sodium (M/T) ratios, equating to up to 10 mol % of total formulation. M/T ratios of BERCLs were normalized to the highest M/T ratio between the formulations. Control lenses were synthesized similarly but without addition of template drug to the pre-polymer formulation. The pre-polymer formulation was vortexed for approximately 1 minute and then mixed in a sonicator for 30 minutes at room temperature to remove dissolved gases and ensure full dissolution of the template drug.

A volume of 65 μL of the pre-polymer formulation was pipetted into polypropylene lens molds (dimensions swollen silicone lens 14.8 mm diameter, 8.4 base curve). Polymerization occurred via UV polymerization using an Omnicure S2000 (Excelitas Technologies Corp., Waltham, MA), with an intensity of approximately 40 mW/cm² for a duration of 2 minutes.

BERCLs were plasma coated in a SPI Plasma Prep III Plasma Cleaner (SPI supplies, West Chester PA), to ensure a hydrophilic surface. Loaded BERCLs were then sterilized via autoclave in their equilibrium solutions in PBS for 30 minutes at 121° C. and stored until use.

The effect of heat and sterilization conditions on bromfenac was also assessed. In 10 mL centrifuge tubes, 3 mL samples of bromfenac solutions of 0.1 mg/mL and 1 mg/ml were heated to 121° C. for 30 minutes. Heated samples were compared to unheated controls via 1H-NMR (400 MHZ, Agilent Technologies, Santa Clara, CA) to ensure that heating process did not result in a change in chemical structure.

### Macromolecular Memory Validation: Drug Binding and In Vitro Microfluidic Release

All lenses were washed following the same procedure and analysis as set forth in Example 2. Dry lens mass was determined in the same manner as demonstrated in Example 2.

Bromfenac in vitro release studies were conducted via a physiological flow microfluidic device in PBS (as shown in FIGS. 3A-3B). The device was produced using poly dimethylsiloxane (PDMS). A mixture of 10:1 ratio of Sylgard 184 silicone base and curing agents was prepared and stirred for 4 minutes, then poured onto a glass plate within a circular mold (55.8 mm diameter). Two needles (1.27 mm outer diameter) were placed into the mold to create an inlet and outlet for flow, and a hemisphere on the glass plate (9.00±0.10 mm radius of curvature) created a well in which contact lenses were placed during release. The device was then cured at 60° C. for 6 hours. For release studies, a drug-loaded lens was placed into the well of the device, then fixed into position with a glass hemisphere (8.75±0.10 mm radius of curvature). The microfluidic device was then sealed onto a glass plate using metal clamps. A kd Scientific Model 220 syringe pump (kd Scientific Inc., Holliston, MA) was used to inject solution through the device at a rate of 3 μL/min, simulating physiological tear flow: Drug release samples were collected and analyzed at different times using UV/Vis spectrophotometry at wavelength of maximum absorption (400 nm).

The templating process during polymerization creates complexation points from multiple polymer chains with drug leading to the creation of memory for drug within flexible polymer chains that comprise the lens structure (as shown and described above see FIG. 1).

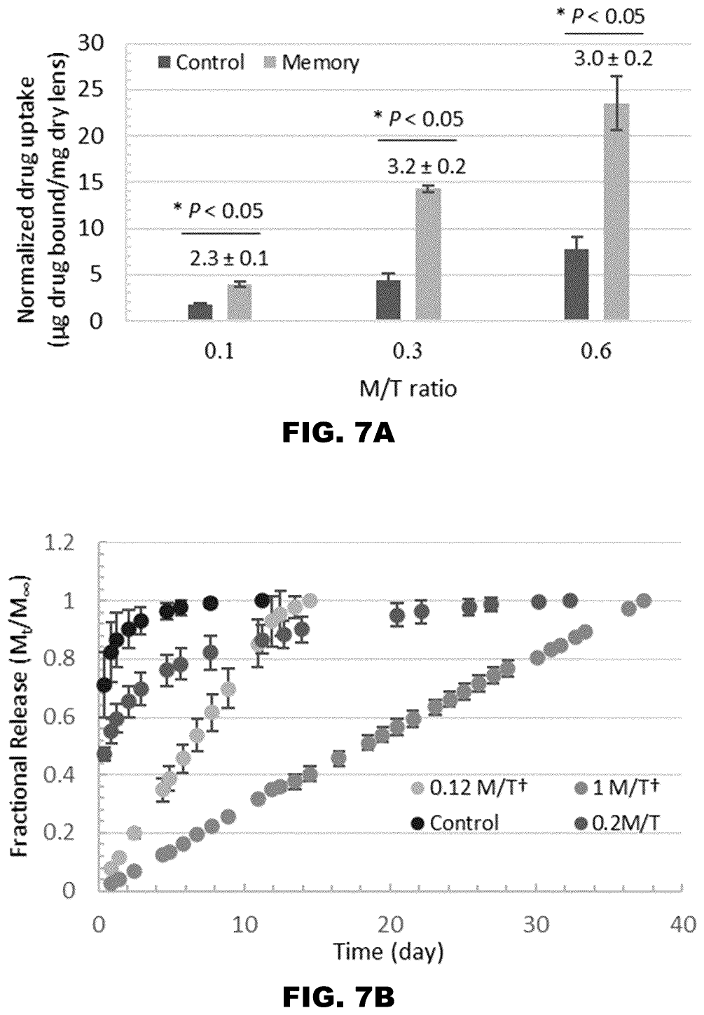

FIG. 7A highlights equilibrium mass binding of bromfenac in BERCLs synthesized at different M/T ratios. Numbers above bars represent imprinting factor, indicating increased memory. BERCLs demonstrated binding values of bromfenac of 3.99±0.30 μg_{drug}/mgₗₑₙₛ, 14.24±0.41 μg_{drug}/mgₗₑₙₛ, and 23.50±2.94 μg_{drug}/mgₗₑₙₛ corresponding to normalized M/T ratios of 0.1, 0.3, and 0.6 and imprinting factors of 2.3±0.1, 3.2±0.2, and 3.0±0.2 compared to corresponding control loading values. The control displayed the lowest drug binding with higher M/T ratio BERCLs exhibiting higher drug loadings supporting the hypothesis that increasing macromolecular memory sites lead to higher drug uptake.

FIG. 7B shows in vitro microfluidic fractional release of bromfenac sodium in PBS versus time for BERCLs synthesized at different M/T ratios. Control lenses released bromfenac very quickly and approximately 70% of their drug payload within 12 hours. With 24 hours, over 85% loaded bromfenac was released with a very small drug amount (0.25 μg or 15%) being released over approximately 3 additional days. This release is expected to be a little better than drug soaking a conventional lens on the market as the controls contain monomers chosen to non-covalently interact with the drug but lack the hypothesized organization of the templated lenses. With an increase in M/T ratio, the release curve shifted to the right and downward towards a more controlled, approaching a zero order or a zero order or constant release rate (i.e. same amount of drug released per unit time). Thus, lenses with increased M/T ratios loaded more bromfenac and released it in a more controlled way for longer periods of time. BERCLs produced with an M/T ratio of 0.2 extended release up to 22 days, with 80% of the payload being delivered by day 8, demonstrating an example of lenses suitable for a week-long continuous therapy.

These results again show that drug loading and release can be altered via modification of the M/T ratio, suggesting that an increase in functional chemistry led to an increase in drug-functional monomer complexation in the pre-polymer formulation and a higher degree of memory site formation in the resulting polymer network.

Polymer volume fraction in the swollen state of BERCLs with M/T ratio of 0.12 were 0.69±0.03 and corresponding control were 0.72±0.09 (FIG. 8A). Equilibrium weight swelling ratio of control lenses were 0.38±0.12 compared to 0.43±0.07 in BERCLs (FIG. 8A). Normalized average molecular weight between crosslinks and mesh size of BERCLs with M/T ratio of 0.12 and corresponding control are highlighted in FIG. 8B. FIG. 8C illustrates the optical transmittance of BERCLs in artificial lacrimal fluid (ALF) and phosphate buffered saline (PBS). FIG. 8D is a picture of a BERCL lens. Structural analysis indicated that BERCLs had a mesh size that was not statistically different than controls, suggesting that formation of macromolecular memory sites lead to extended release and increased drug loading in templated lenses rather than a tighter polymer architecture or smaller mesh size. An increase in macromolecular memory site formation alters the orientation of polymer chains without affecting structural properties such as mesh size and molecular weight between crosslinks. However, there is a synergistic relationship between mesh size and imprinting efficiency as previously studied by our group.

As mesh size and the flexibility of the network structure decrease, macromolecular memory increases. To analyze this, lenses were produced with a higher elastic modulus and decreased mesh size by keeping the formulation the same but utilizing macromers of the same chemistry that were ˜13 times lower in molecular weight/smaller in linear size. FIG. 7B highlights that lenses with a modulus of 2.3+/−0.2 MPa and a normalized mesh size of 0.35 exhibited zero order release or constant release rate (M/T of 0.12 and 1 data sets). At similar normalized M/T ratios (0.12 and 0.2 data sets in FIG. 7B), the synergy between mesh size and memory becomes apparent as the moduli was 3.8 times higher and mesh size 2.4 times lower for the M/T 0.12 data set which displayed more zero-order release even at a slightly lower M/T ratio. Lenses with an M/T of 1 with an increased modulus and tighter average network structure displayed a zero order release profile with payload delivered in 37 days. Additionally, for M/T values of 0.12 and 1, the surface of the lenses were quickly rinsed before placing in the microfluidic device to simulate the procedure for ocular placement. This directly led to less drug being released in the first 12 hours from drug adsorbed to the surface or close to the surface that did not interact with many memory sites (comparing to M/T of 0.2, which were not rinsed).

### Optical Property Analysis

Optical clarity studies were conducted by analyzing transmittance of visible light (450-700 nm) through circular hydrogel segments, cut with a cork borer with a diameter of 1.5 mm. Each lens segment was placed in the bottom of a 96 well plate and hydrated in 200 μL of PBS or artificial lacrimal fluid (ALF) (6.78 g/L NaCl, 2.18 g/L NaHCO₃, 1.38 g/L KCl, 0.084 g/L CaCl₂·2H₂O, pH 8) along with a blank well containing only 200 μL of PBS or ALF, with care taken to ensure that there were no air bubbles present in any wells. Absorbance values of each well was measured in a Tecan Infinite M200 Pro spectrophotometer (Tecan, Männedorf, Switzerland) and absorbance values of blank wells were subtracted from wells containing lenses. Refractive index of contact lenses swollen in both PBS and ALF was measured using an IP65 digital refractometer (Sper Scientific, Scottsdale, AZ).

### Contact Angle and Specific Gravity Analysis

Contact angle with water was measured by cutting circular segments (5 mm diameter) from BERCLs using a cork borer. Using a micropipette, a water droplet was placed on the surface of lens hydrogel cutouts and analyzed using a surface contact angle goniometer (Theta Flex Tensiometer, Nanoscience Instruments, Phoenix, AZ) via sessile drop analysis.

To analyze specific gravity, lenses were first dried in a vacuum oven (T=30° C., 28 in. Hg vacuum) until no change in mass was observed (<0.1% change in mass). Density of dried lenses and lenses swollen in PBS or ALF were measured using a VWR analytical balance (VWR, Radnor, PA) with a density analysis kit using hexane for non-solvent liquid measurement.

Elastic modulus determination, oxygen transport analysis, and hydrogel structural analysis were conducted using the same processes as described in Example 2. Analyses and results are shown above in Example 2.

### Rabbit Purchase and Handling

Male New Zealand white rabbits, weighing between 3 and 4 kg were purchased from Myrtles Rabbitry (Thompsons Station, TN). Upon arrival, rabbits were acclimatized for at least 7 days to reduce stress and achieve psychological, nutritional, and physiological stability. All animal facilities used in this project were certified and inspected by AAALAC and the USDA. All rabbits were treated according to the ARVO Statement for the Use of Animals in Ophthalmic and Vision Research and NIH standards. Prior to experimental work, protocols were reviewed and approved by the Cooper Hospital Institutional Animal Care and Use Committee (IACUC). Prior to experimental manipulation, animals were handled on a regular basis and acclimated via non-threatening interaction (removing from cage, petting, feeding treats). Animals were housed in individual cages in a light controlled room with a 12-hour light/dark cycle and temperature and humidity of 21±1° C. and 40±5% respectively, with no restriction of food and water intake.

Animal restraint occurred via wrapping with a towel with single person manual restraint during experimental procedures, with no need for an additional, commercial restraint device. Rabbits in restraint were also continuously monitored. Prior to experimentation, all animals were acclimated to manual restraint at increasing time intervals until the maximum time of restraint was achieved with no distress to the rabbit. Rabbits were housed in stainless steel cages with slatted floor provided by Cooper Hospital Division of Laboratory Animal Health.

### Pre-Study Lens Fitting and Acclimation

Once the rabbits had undergone the standard acclimation period, a complete ophthalmic examination was performed by a board-certified veterinary ophthalmologist to ensure no ocular abnormalities were present that could interfere with the study. Examination included neuroophthalmic exam, Schirmer 1 tear test (without anesthetic) measuring baseline and reflex tear secretion for 1 minute with strips (Merk, Summit, NJ), rebound tonometry (Tonovet, ICare, Finland), fluorescein staining (BioGlo, Madhu Instruments, India), slit lamp biomicroscopy (Kowa SL-17, Torrence, California) and complete fundic examination with indirect ophthalmoscopy (Vantage Pus, Keeler, Malvern, PS: with Volk 28 and 20 diopter condensing lens, Mentor, OH) after pharmacologic mydriasis was achieved with Tropicamide 1% solution (Akorn, Lake Forest, IL).

Prior to administration of a BERCL, rabbits were given non-therapeutic control lenses to assess fit and acclimatize animals to wearing a lens. Rabbits were given a non-therapeutic lens in the right eye in order to determine lens fit and comfort. Non-drug loaded lenses were worn for 24 hours, and at the end of the 24-hour period animals were examined for redness, ocular swelling, or any signs of discomfort. For the duration of lens wear, animals wore Elizabethan collars to prevent removal of the lens as well as self-trauma.

### Lens Administration

Sterile BERCLs were removed from their vials, and commercially available, sterile, saline solution (Ocu Fresh, Optics Laboratory, Inc.) was applied to remove the excess of bromfenac on the lens surface and the lens was placed on the right cornea of each rabbit. All rabbits were sedated with dexmedetomadine (100 mg/kg intramuscular injection, Dexdomitor, Orion Corporation, Finland) to allow placement of the BERCL (8.4 base curve and 14.8 mm OD) and temporary tarsorrhaphy to prevent early extrusion of the BERCL and to prevent drying of the lens edges on the ocular surface. This procedure was performed without complication in all rabbits, and no nictitating membranectomy was needed. One to two tarsorrhaphy sutures were placed at the lateral eyelid margin with 5-0 silk (Perma-Hand, Ethicon, San Lorenzo, PR) in a simple horizontal mattress pattern. Sedation was reversed with atipamazole (200 mg/kg intramuscular injection, Antisedan, Orion Corporation, Finland). Recovery from sedation was without complication. All rabbits were ambulatory within 3 minutes of reversal and began eating and drinking one hour after sedation. An Elizabethan collar was placed to prevent self-trauma.

Both the BERCLs and tarsorrhaphy were tolerated well by all rabbits. The Elizabethan collar remained in place for the entire study period. At the completion of the study, the tarsorrhaphy sutures were removed to facilitate a complete ophthalmic examination. No sedation was needed to remove the tarsorrhaphy sutures or contact lenses.

### Example 4

### In Vivo Bromfenac Release and Analysis

The synthesis materials and methodology of silicone hydrogel contact lenses as described in Example 3 were followed for bromfenac release and analysis. During the first 24 hours of BERCL wear, tear film samples were taken from the lower eyelid and conjunctival cul-de-sac at 12 hours and 24 hours via 5 μL disposable glass capillary tubes. Tear samples were taken in volumes of 2-5 μL. Tear samples earlier than 12 hours could not be taken as anesthetics used during the tarsorrhaphy caused decreased lacrimation within animals. At 12 hours, 24 hours, and each day after, 1 tear sample was taken from each animal each day for the duration of wear. BERCLs were worn for 8 days total, after which sutures and BERCLS were removed. Each day during tear sampling, animals were closely examined for any ocular redness, protein buildup, swelling, and signs of discomfort.

Ocular bromfenac concentration was measured via spectroscopic analysis using a Nanodrop spectrophotometer (Thermo Fisher Scientific, Waltham, MA) with 8 analysis pedestals. In order to precipitate ocular protein out of tear samples, 2 μL of each sample was treated with 20 μL of 6.0 N hydrochloric acid and 40 μL of DI water. Samples were centrifuged at 13000 RPM and 4° C. for 10 minutes. Eight supernatant samples from the treated tear samples were collected and analyzed via the nanodrop at a wavelength of 280 nm. Tear samples with no applied bromfenac were taken prior to administration of therapeutic lenses. Protein extraction and spectroscopic measurement was performed on these samples, which were used as blanks for samples with drug.

At the conclusion of the study, BERCLs were removed from each animal. Mass of drug remaining in each BERCL was determined by swelling each lens in a known amount of ethanol. The supernatant ethanol solution was measured using UV/Vis spectroscopy (400 nm) to determine the mass of bromfenac in solution.

In order to assess pharmacokinetics of the topical eye drop, rabbits were given one ˜40 μL bromfenac sodium eye drop (0.09%, Bromday™) in the right eye. Following administration of the eye drop, tear samples (2-5 μL) were taken via 5 μL disposable glass capillary tubes. Tear samples were taken at 15 minutes, 30 minutes, 45 minutes, 60 minutes, 75 minutes, and 120 minutes. Once bromfenac was not observed in the tear film concentration, no additional tear samples were taken.

The areas under the concentration-time curve (AUC) and the area under the first moment of the concentration versus time curve (AUMC) were calculated according to the linear trapezoidal rule. The mean residence time (MRT) was calculated as the ratio between the AUMC and the AUC.

### Histological Analysis and TUNEL Assay

Rabbits were enucleated and globes fixed in 10% neutral buffered formalin. Corneas were then isolated and cut in half using surgical scissors. Corneal slices were placed in a cassette and processed using a Tissue-Tek VIP 6 Automatic Processor (Sakura Finetek, Inc., Torrance, CA). Corneal slices were then embedded in parrafin wax (Parrafin Histoplast PE, Thermo Fischer Scientific, Carlsbad, CA) using a Tissue-Tek TEC5 Embedding Station (Sakura Finetek, Inc). Five micron sagittal sections were prepared using a microtome (Shandon, Finesse 325 W Low Profile Rotary Microtome: Thermo Fischer Scientific) and slides were dried overnight at room temperature. Hematoxylin and Eosin (H&E) and Periodic-Acid Schiff (PAS) stainings were performed and sections visualized by microscopy (Zeiss, Axio Observer, Carl Zeiss, Inc, White Plains, NY).

Paraffin embedded corneal sections (5 μm) were prepared as described and TUNEL assay was performed (Biotium, 89410-738, Fremont, CA). Sections were deparaffinized and dehydrated according to standard protocols. Slides were then washed twice in 1×PBS (5 min each) and sections permeabilized with 1 μg/mL proteinase K/10 mM Tris, pH=7.5. Slides were washed twice in 1× PBS (5 minutes each). Positive control sections were then treated with 200 μg/mL DNaseI/water for 10 min at room temperature and washed twice with IX PBS (5 min each). Sections were incubated with TUNEL Equilibration Buffer for 5 minutes at room temperature, followed by incubation with TUNEL reaction mix (1 μl TdT-CF640R+50 μl TUNEL Reaction Buffer) and incubated in a humidified 37° C. chamber for 1.5 hours. Negative control sections were incubated with only TUNEL Reaction Buffer (no TdT). Slides were washed three times with 1× PBS (5 minutes each), counterstained with Hoescht-33342 and mounted in VECTASHIELD® Mounting Medium (H-1400, Vector Laboratories Inc, Burlingame, CA). Microscopic images were obtained using a Zeiss Axio Observer Microscope (Carl Zeiss, Inc, White Plains, NY).

### Statistical Analysis

M/T ratios of BERCLs were normalized to the highest M/T ratio between the formulations. Average molecular weight between crosslinks and mesh size of BERCLs were normalized to the highest value between formulations. Outliers were evaluated via two-tail t-test with P>0.05 considered not significant. Results are presented as mean±SD with n≥3.

### Pre-Study Ophthalmic Evaluation

Slit lamp examination was overall unremarkable for all rabbits prior to the study. Schirmer tear test values were normal for all rabbits (12.0±2.5 mm/min right eye, 11.7±2.1 mm/min left eye) and the pre-corneal tear film was deemed of adequate quality, normal in appearance and was free of any debris. Additionally, no abnormalities were observed in the palpebral or bulbar conjunctiva, along the meibomian glands of the eyelid margin or within the nasolacrimal system. Careful examination with both direct and indirect illumination of the corneal epithelium, corneal stroma and endothelium did not reveal any abnormalities. The cornea was free of blood vessels, edema, deposits, or any other opacities. Fluorescein staining was performed and the cornea was evaluated with a cobalt blue filter via indirect illumination with the portable slit lamp. Corneal staining using the Cornea and Contact Lens Research Unit (CCLRU) grading system was recorded as 0/4 (absent) in all rabbits prior to placement of the contact lens.

No biomicroscopic evidence of anterior uveitis was seen (no aqueous or cellular flare), and the lens of all rabbits in the study was normal with no opacities present. The iris was normal in color with normal vascularization and thickness.

Rebound tonometry was performed with the Tonovet (ICare, Finland), and values were within the normal range in all rabbits (10.2±2.7 mmHg right eye, 12.7±2.9 mmHg left eye).

Fundic examination performed with indirect ophthalmoscopy revealed no significant clinical abnormalities in the vitreous, retinal arterioles or venules, choroidal vasculature, neurosensory retina or optic nerve. While normal visual testing (menace response) is difficult in rabbits, no visual abnormalities were seen when observing the rabbits in their normal environment. All rabbits were deemed excellent candidates for the contact lens study based on their normal ophthalmic examination.

### In Vivo Dynamic Release Compared to Topical Therapy

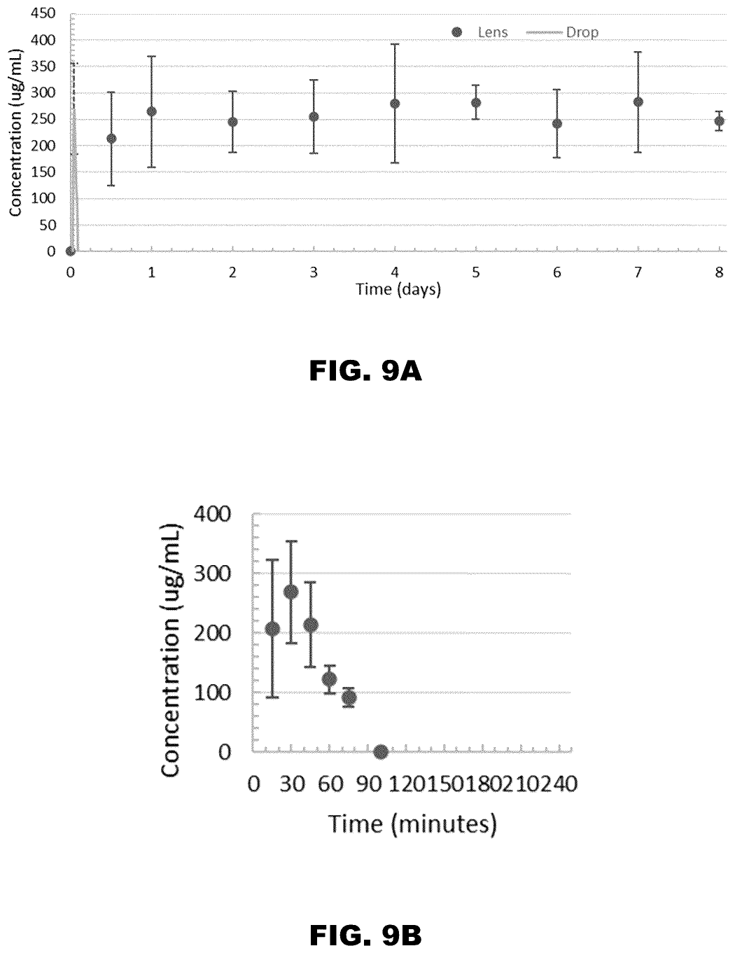

FIGS. 9A-9B and Table 3 shows the dynamic in vivo tear film concentration profile of bromfenac in rabbits from BERCLs compared to the topical administration of the commercially available Bromday™ (bromfenac ophthalmic solution, 0.09%, Bausch+Lomb). The concentration of applied topical drops quickly reached a Cₘₐₓ of 269.3±85.7 μg/mL within 30 minutes of application, and exponentially decreased from the tear fluid within 100 minutes, indicating that the entirety of the instilled drug had been removed from the precorneal area due to tear turnover (FIG. 9B). Table 3 shows parameter comparison of BERCLs and topical drops. The bioavailability (AUC) and mean residence time (MRT) were compared. The AUC unit (μg wk/mL), wk represents duration of study of 8 days. AUC₀₋₈ days for topical drops assumes 1 drop/day, for 2 drops/day AUC₀₋₈ days is 155.2. All tests were performed with four to six replicates, errors bars represent mean±SD. Table 3 shows that the AUC and MRT of BERCLs were 26 and 155 times higher than the topical eye drops with a recommended dosage of 1 drop/day.

TABLE 3

| | | | AUC_{0-24 hrs} | AUC_{0-8 days}* | |
|---|---|---|---|---|---|
| Delivery | Cₘₐₓ | tₘₐₓ | (μg | (μg | MRT |
| mechanism | (μg/mL) | (min) | day/mL) | wk/mL)^{†} | (hr) |
| Lens | 282.4 ± 95 | 10080 | 172.6 | 2012.3 | 100.7 |
| 1 Drop | 269.3 ± 85.7 | 30 | 9.7 | 77.6 | 0.65 |
| 2 Drops | 269.3 ± 85.7 | 30 | 19.4 | 155.2 | 0.65 |

The concentration of bromfenac in the tear fluid from the applied BERCL reached a bromfenac concentration of 213.1±88.3 μg/mL within 12 hours of application and maintained an average ocular concentration of 256.4±23.1 μg/mL (Cₘₐₓ of 282.4±95 μg/mL) for the duration of the 8-day study. Average tear concentration values of bromfenac from the contact lenses were statistically similar compared to the commercial eye drop group indicating that a clinically effective and therapeutic concentration was reached. Ocular tear bromfenac concentration from the BERCL was relatively constant over an 8-day period of continuous night and day lens wear, indicating that this method of treatment can deliver a much more consistent dosage of drug than eye drops. This demonstrates a steady concentration of drug being maintained in the tear film for duration of wear via release from an extended-wear silicone hydrogel contact lens.

The mean residence time (MRT) of BERCLs was calculated to be 100.7 hours or 4.2 days while drops displayed an MRT of 0.65 hours or 39 minutes, resulting in 154.9 times increase in MRT with lenses compared to drops. Bioavailability of BERCLs (AUC₀₋₈ days) was calculated to be 2,012.3 (μg day/mL), which was 26 times greater than the topical eye drops if applied once a day following the recommended dosage regimen. The lenses were removed after the last tear samples on the 8^{th} day, and the average mass of bromfenac left within the lens at the conclusion of the study was 1.8±0.2 μg, indicating that BERCLs released the majority of their drug payload during 8 day wear. With macromolecular memory, release rate and loading can be controlled to deliver a therapeutic concentration for a period of time delivering close the entire loaded drug payload, parameters vital for commercialization.

White New Zealand rabbits and humans have similar lacrimation rates (rabbit 7.6+/−2.3 mm/min, human 6.3+/−1.2 mm/min, Schirmer 1-2 min), lacrimal volumes (rabbit 7.5±2.5 μL, human 7 μL), and tear film thicknesses (rabbit 45 μm, human 41-46 μm), but differ in tear film break-up times and eye blink frequency (rabbit ˜once every 10 min, human once every 5 sec). With different memory ratios, lenses can be produced with higher and lower release rates. By utilizing memory mechanisms, the release rate, and thus the clinical concentration reached, can be altered by changing the engineering design of such systems.

### Post-Study Ophthalmic Evaluation

Results of the post-study examination were similar to the pre-study exam. The temporary tarsorrhaphy suture(s) were removed and BERCLs were subsequently removed. Schirmer tear test values and intraocular pressure measurements remained normal OU in all rabbits. Complete slit lamp biomicroscopy was performed using the same method as the pre-study examination. The contact lens was still in place in all rabbits except one, in which the tarsorrhaphy was broken and the lens had exited the eye. It was documented that the lens was in place in this rabbit one day prior to exam. Contact lens fit remained appropriate with normal surface appearance and wetting of the lens. No deposits were observed on the contact lens itself. In one rabbit the lens had ripped slightly during the study and a small superficial corneal abrasion was present in inferior nasal portion of the eye (CCLRU grading Type 3, Depth 1, and Extent of Surface Involvement 1). All other rabbits retained the contact lens until the post study exam. No fluorescein retention was present in any of the remaining rabbits (CCLRU grading 0/4). Once the contact lens was removed, the cornea was again examined in detail using slit lamp biomicroscopy.

Other than the previously documented corneal abrasion in the rabbit in which the lens had torn, no abnormalities were observed. The pre-corneal tear film was again deemed adequate and of normal quality. No corneal vascularization, deposits or edema was seen in any of the rabbits once the contact lens was removed. No biomicroscopic evidence of anterior uveitis was seen with no aqueous or cellular flare, and fundic examination remained normal in all rabbits. This example demonstrated that the BERCL was well tolerated by all animals in the study. The contact did not have a negative effect on tear production or overall corneal health. All rabbits appeared comfortable with good retention of the contact lens.

### Histological Analysis

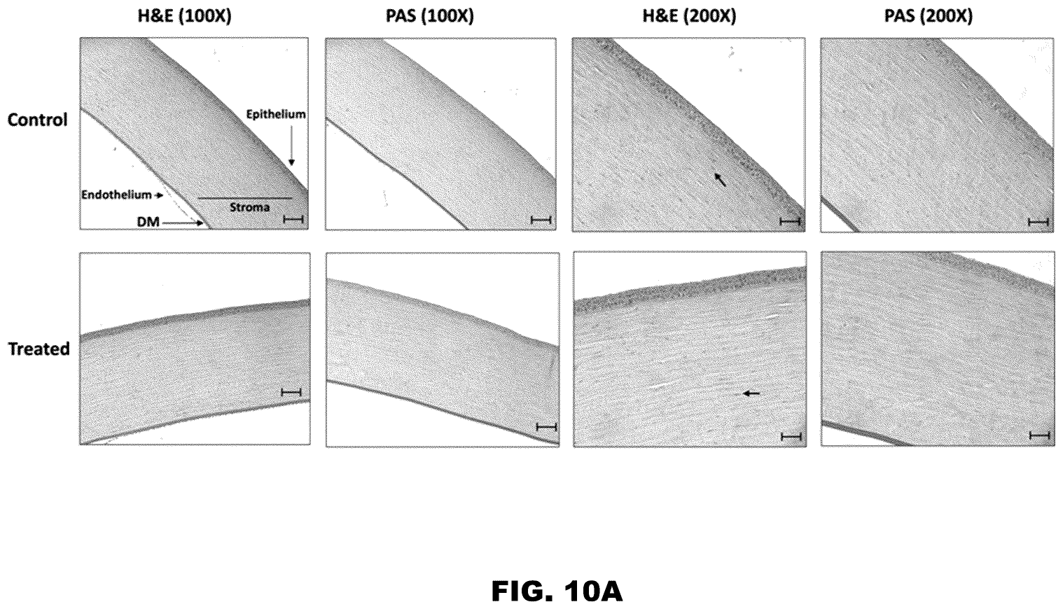

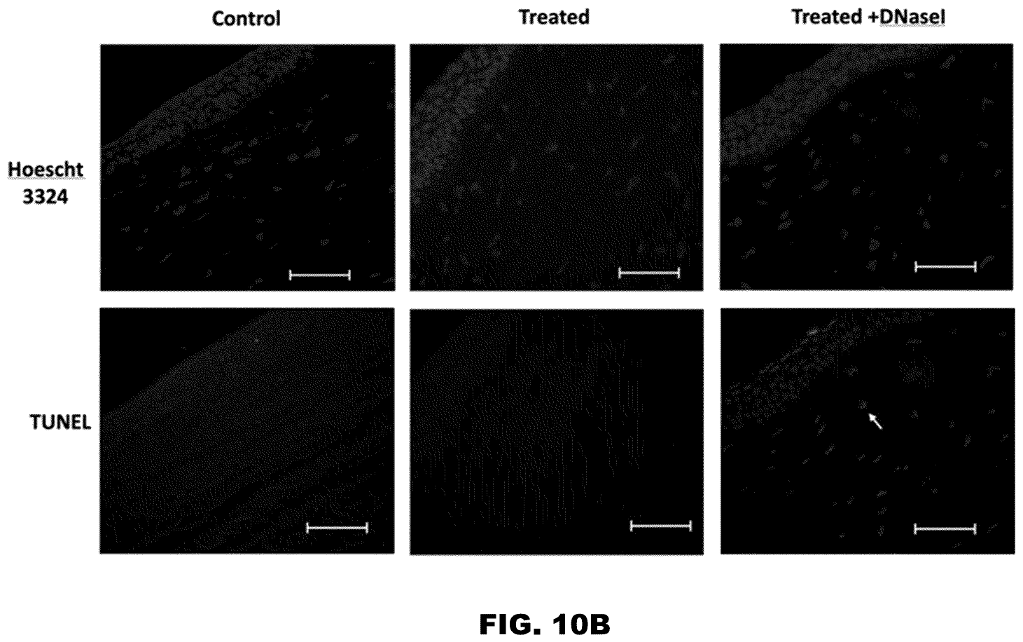

FIGS. 10A-10B illustrate histological analysis of ocular tissue treated with bromfenac extended-release contact lenses (BERCLs) compared to control tissue with no lens or drug. The left eye was untreated (control) and the right eye was treated with a BERCL. At the end of the study (8 days), rabbits were euthanized and eyes were enucleated and fixed in 10% neutral buffered formalin. Whole eyes were dissected to isolate corneas which were then placed in cassettes and processed. Processed corneas were embedded in paraffin and 5 μm sections generated. In FIG. 10A, sections were stained with hematoxylin and eosin (H&E, staining nuclei purplish-blue and extracellular matrix and cytoplasm pink) and Periodic-Acid Schiff (PAS, indicating lymphocytes and mucopolysaccharides via magenta color). DM=Descemet's membrane: arrow=keratocyte. In FIG. 10B. TUNEL assay performed in order to detect DNA fragmentation that occurs in the late stage of apoptosis. Treated tissue samples were incubated with DNaseI as a positive control for the TUNEL assay. The arrow indicates DNA fragmentation visible in keratocytes. Lenses were well tolerated with no adverse events. No histological difference was observed between control and treated samples which contained the drug releasing lens for over 8 days indicating that BERCLs were safe to wear. There was no evidence of hyperplasia or hypertrophy, no change in keratinocyte number, nor immune cell infiltration in the treated samples compared to control samples, indicating that lenses worn for 8 days releasing drug have a similar safety profile to ocular tissue with no lens or drug. Scale bar=100 μm. All tests were performed with six replicates.

Histological analysis determined that corneas from animals with applied BERCLs showed none of the common signs of corneal injury and appeared to be normal (FIG. 10A). The epithelium, stroma, endothelium and Descemet's membrane were all intact and visibly normal: there was no evidence of epithelial hyperplasia or hypertrophy. There were no changes observed in keratinocyte number or morphology with treatment. In addition, there were no signs of inflammation, including no increase in Goblet cell and lymphocyte infiltration.

TUNEL stain results shown in FIG. 10B demonstrate no presence of apoptotic epithelial corneal cells nor keratocytes in treated corneal samples. In addition, H&E staining showed no evidence of pathology typical for corneal injury, including vascularization, acellular fibrotic deposits, lack of cuboidal cells in epithelial basal layer, infiltrate of immune cells, or areas devoid of keratocytes.

### Example 5

### Exemplary Treatment Strategies Using Contact Lens Delivery Systems

The various contact lens delivery systems described herein have numerous suitable applications of use for treatment. New treatment strategies for post-cataract, uveitis, post-LASIK or other forms of laser-assisted ocular surgery, and corneal abrasion treatment are shown in Table 4 below. Utilizing a first-line NSAID, bromfenac sodium, and matching typical patient recall times, an efficient treatment strategy to treat inflammation and pain has high potential to replace a regimen of numerous topical eye drops and increase efficacy.

Table 4. Treatment Strategies of Bromfenac Extended-Release Contact Lenses (BERCLs) Compared to Topical Eye Drops. The treatment strategies are based on clinical standard of care with BERCLs regimen based on in vivo release results.

TABLE 4

| | | | Bromday ™ | BromSite ® | |
|---|---|---|---|---|---|
| | | | Bromfenac | Bromfenac | |
| | | | Ophthalmic | Ophthalmic | Bromfenac |
| | | | Solution | Solution | Extended |
| | | | (0.09%) | (0.075%) | Release Lens |
| | | | Topical | Topical | (BERCL) |
| | | Treatment | Regimen | Regimen | (1 lens/7 days |
| Treatment | Indications | Duration | (1 drop/day) | (2 drops/day) | or 1 week) |
| Post- | Treatment of | 2-6 weeks | 14-42 drops | 28-84 drops | 2-6 lenses |
| Cataract | Post- | | | | |
| Surgery*⁺ | operative | | | | |
| | Inflammation | | | | |
| | & Pain | | | | |
| Anterior | Treatment of | 6-10 weeks | 42-70 drops | 84-140 drops | 6-10 lenses |
| Uveitis* | Inflammation | | | | |
| Post- | Treatment of | 1 week_{†} | 7 drops | 14 drops | 1 lens |
| LASIK_{‡} | Post- | | | | |
| | operative | | | | |
| | Inflammation | | | | |
| | & Pain | | | | |
| Corneal | Treatment of | 1 week_{†} | 7 drops | 14 drops | 1 lens |
| Abrasion** | Inflammation | | | | |
| | & Pain | | | | |

Table 4 is based on clinical standard of care with BERCLs regimen based on in vivo release results. In post-cataract surgery (*) and anterior uveitis (*) treatments, lens wear time of 1 week/7 days matches ophthalmologist standard of care recall or patient follow-up. Additionally in post-cataract surgery (+), a dropless or lens releasing bromfenac (NSAID) delivery strategy will include a single-dose of a broad-spectrum antibiotic (e.g., moxifloxacin) via intracameral irrigation (during surgery) or injection (subconjunctival or sub-Tenon's injection, or intravitreal). Antibiotic irrigation/injection are becoming more widely accepted and a promising substitute for standard eye drop therapy significantly reducing prophylactic endophthalmitis risk, with intracameral irrigation reducing risk 6-7 times. Post-LASIK (‡, laser-assisted in situ keratomileusis) includes other forms of laser-assisted ocular or vision surgery were analyzed such as PRK (photorefractive keratectomy) surgery, SMILE laser surgery, Epi-LASIK (epithelial-LASIK) surgery, lens replacement surgery or refractive lens exchange, LASEK (laser epithelial keratomileusis) eye surgery, PresbyLASIK or multifocal LASIK. For Post-LASIK(+) and corneal abrasion (+), typical treatment is 1 week/7 days with laser-assisted surgery and superficial abrasions, but complications and deeper abrasions may require an additional week after follow-up. Further, for minor corneal abrasions(**), healthy cells quickly fill the defect to prevent refraction irregularity and infection, and deeper abrasions can lead to corneal scarring with scarring leading to corneal transplant.

This example of clinical treatment applications with a lens releasing bromfenac (NSAID) strategy can include a single-dose delivery of a broad-spectrum antibiotic with or without a long acting SAID (e.g., moxifloxacin or triamcinolone acetonide-moxifloxacin, dexamethasone-moxifloxacin) via intracameral irrigation (during surgery) or injection, subconjunctival or sub-Tenon's injection, or intravitreal depot placement. These irrigations and/or injections are becoming more widely accepted and a promising substitute for standard eye drop therapy. With intracameral antibiotics compared to topical eye drops, endophthalmitis risk was reduced 6 to 7 times. However, to prevent post-operative endophthalmitis, an appropriate volume and concentration of moxifloxacin is needed considering the aqueous fluid turnover. Dropless surgery with an NSAID releasing lens, as described herein with the contact lens delivery systems, offers better compliance for control of inflammation, swelling, pain, and prophylaxis against infection. With controlled release of NSAID, it also offers the benefit of not using steroids (SAID) for effective treatment for patients without increased risk for endophthalmitis.

It is to be understood that while the embodiments of the disclosure have been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure. All publications discussed and/or referenced herein are incorporated herein in their entirety.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. A contact lens delivery system containing ocular therapeutic agents, comprising:
a silicone hydrogel contact lens comprising a cross-linked polymeric hydrogel matrix comprising functional monomers and low molecular weight crosslinking agents, and
wherein the cross-linked polymeric hydrogel matrix has macromolecular memory sites that complex an ocular therapeutic agent and release the ocular therapeutic agent from the hydrogel matrix over time while in contact with a surface of an eye,
wherein the cross-linked polymeric hydrogel matrix contains an effective amount of the at least one ocular therapeutic agent, and
wherein the silicone hydrogel contact lens are afocal, multi-focal, vision correcting or non-correcting, plano, or bandage lenses having no vision correction and having an elastic modulus between about 0.5 mPa and about 2.0 MPa.

2. The composition of claim 1, wherein (i) the functional monomer is a silicone-based monomer, (ii) the functional monomers or an additional monomer are selected from the group consisting of N,N-dimethylacrylamide (DMA), 2-hydroxyethylmethacrylate (HEMA), hydroxyethyl acrylate, hydroxypropyl acrylate, hydroxypropyl methacrylate (HPMA), trimethylammonium 2-hydroxy propylmethacrylate hydrochloride, dimethylaminoethyl methacrylate (DMAEM), diethyl aminoethyl methacrylate (DEAEM), diallyl dimethyl ammonium chloride (DADMAC), methacrylamidopropyltrimethyl ammonium chloride (MAPTAC), dimethylaminoethyl-methacrylamide, acrylic acid, methacrylic acid, acrylamide, methacrylamide, allyl alcohol, vinylpyridine, glycerol methacrylate, N-(1,1 dimethyl-3-oxobutyl)acrylamide, N-vinyl-2-pyrrolidone (NVP), acrylic acid, methacrylic acid, N-vinyl piperidone, N-vinyl caprolactam, N-vinyl-N-methyl acetamide, N-vinyl formamide, N-vinyl acetamide, N-vinyl-N-methyl acetamide, N-vinyl-N-ethyl acetamide, N-vinyl isopropylamide, N-vinyl-N-ethyl formamide, silicone-based monomer or macromer, and combinations thereof, or (iii) the functional monomer or an additional monomer is a is polysiloxane, polydimethyl siloxane, tris(trimethylsiloxy)silyl propyl methacrylate (TRIS), a hydrophilic TRIS derivative, monomer or macromer with pendent silicone groups, Lotrafilcon A or B, Betacon macromer, a macromer comprising two terminal methacryloxyethyl and/or methacryloxypropyl terminated groups and at least two polysiloxane or polydimethylsiloxane segments, or combinations thereof.

3. (canceled)

4. (canceled)

5. The composition of claim 1, wherein the low molecular weight crosslinking agents are selected from the group consisting of: polyethylene glycol (200) dimethacrylate (PEG200DMA), ethylene glycol dimethacrylate (EGDMA), tetraethyleneglycol dimethacrylate (TEGDMA), N,N′-Methylene-bis-acrylamide, polyethylene glycol (600) dimethacrylate (PEG600DMA) and combinations thereof.

6. The composition of claim 1, wherein the ocular therapeutic agent is at least one drug, and wherein the drug is selected from the group consisting of an antibiotic, an anti-inflammatory, an antihistamine, an antiviral agent, a cancer drug, an anesthetic, a cycloplegic, an anticholinergic, an antimuscarinic, a mydriatics, a lubricant agent, a hydrophilic agent, a decongestant, a vasoconstrictor, vasodilator, an immuno-suppressant, an immuno-modulating agent, an anti-glaucoma agent, an anti-infective, hyperosmolar agent, vitamins, growth factors, growth factor antagonists, sympathomimetics, an adrenergic agonist, an anti-cataract agent, an anti-hypertensive agent, an anti-macular degeneration agent, an ocular permeation enhancing agent, an anti-retinal disease agent, an anti-retinitis pigmentosa agent, an anti-diabetic retinopathy agent, an ocular myopia controlling agent, an ocular diagnostic agent, and combinations thereof.

7. (canceled)

8. The composition of claim 6, wherein (i) the drug is an anti-inflammatory drug selected from the group consisting of triamcinolone acetonide, dexamethasone, dexamethasone sodium phosphate, and other corticosteroids, bromfenac sodium, diclofenac sodium and other non-steroidal anti-inflammatory drugs (NSAIDs), or (ii) wherein the drug is an antibiotic selected from the group consisting of moxifloxacin or other quinolone or fluoroquinolone antibiotics, cefuroxime and other cephalosporin antibiotics, vancomycin and other glycopeptide antibiotics including teicoplanin, telavancin, ramoplanin and decaplanin, corbomycin, and complestatin.

9. (canceled)

10. The composition of claim 5, wherein the cross-linked polymeric hydrogel matrix contains from about 20 μg to about 500 μg of the drug.

11. The composition of claim 1, wherein the cross-linked polymer hydrogel matrix comprises in an amount of from about 50 wt-% to about 90 wt-% silicone or siloxane monomers or macromers, methacryloxypropyl-tris-(trimethylsiloxy) silane (TRIS), and/or N,N dimethyl acrylamide (DMA).

12. The composition of claim 1, wherein the crosslinking agents comprise ethylene glycol dimethacrylate (EGDMA) and/or polyethylene glycol 200 dimethacrylate (PEG200DMA) in an amount of from about 0.5 wt-% to about 15 wt-%.

13. The composition of claim 1, wherein the cross-linked hydrogel matrix comprises a photo-initiator in an amount of from about 0.01 wt-% to about 5 wt-% and/or a solvent comprising in an amount of from about 0 wt-% to about 60 wt-%.

14. The composition of claim 1, wherein the cross-linked polymeric hydrogel matrix is formed by the steps of generating a solution comprising amounts of the ocular therapeutic agent and functional monomer, complexing the functional monomers and the ocular therapeutic agent through non-covalent interactions, initiating copolymerization of the functional monomers and the low molecular weight crosslinking agents to form the memory sites, optionally washing the hydrogel matrix, and loading the ocular therapeutic agent into the memory sites.

15. The composition of claim 1, wherein the silicone hydrogel contact lens has an oxygen permeability of at least about 50 Barrer.

16. A method of treating one or both eyes of a mammal in need thereof, comprising: contacting the contact lens delivery system according to claim 1 to one or both eyes of a mammal to provide controlled release of the at least one ocular therapeutic agent for a duration of treatment.

17. The method of claim 16, wherein (i) the one or both eyes of the mammal are in need of treatment with steroidal anti-inflammatory drugs (SAIDs) and/or non-steroidal anti-inflammatory drugs (NSAIDs), or (ii) wherein the one or both eyes of the mammal are in need of treatment with an antibiotic drug therapy.

18. (canceled)

19. The method of claim 16, wherein the contacting of the contact lens delivery system is continuously for a duration of treatment less than about 30 days, or between about 1 week to about 15 weeks, and wherein the silicone hydrogel contact lens are replaced every about 5 to about 10 days throughout the duration of treatment, or about 7 days throughout the duration of treatment.

20. (canceled)

21. (canceled)

22. The method of claim 16, wherein the controlled release of the at least one ocular therapeutic agent is approaching a zero order or a zero order release rate.

23. The method of claim 22, wherein the effective amount of the at least one ocular therapeutic agent is increased or decreased by replacing the silicone hydrogel contact lenses with another silicone hydrogel contact lenses having the increased or decreased effective amount of the at least one ocular therapeutic agent.

24. The method of claim 16, wherein the silicone hydrogel contact lens are used for the treatment of a condition selected from the group consisting of post-cataract surgery, post-laser-assisted in situ keratomileusis (LASIK) or other forms of laser-assisted ocular and/or vision surgery, uveitis, and corneal abrasion, and wherein the other forms of laser-assisted ocular and/or vision surgery are selected from the group consisting of photorefractive keratectomy (PRK) surgery, small incision lenticule extraction (SMILE) laser surgery, epithelial-LASIK surgery, lens replacement surgery or refractive lens exchange, laser cataract surgery, laser epithelial keratomileusis (LASEK) surgery, PresbyLASIK surgery, and multifocal LASIK surgery.

25. (canceled)

26. The method of claim 24, wherein the condition is (i) post-cataract surgery, and wherein the duration of treatment is between about 1 week and about 8 weeks, and wherein the silicone hydrogel contact lens are replaced every about 5 to about 18 days throughout the duration of treatment, or every about 7 days, (ii) post-LASIK or other forms of laser-assisted vision surgery, wherein the duration of treatment is between about 5 days and about 3 weeks, and wherein the silicone hydrogel contact lens are replaced every about 5 to about 10 days throughout the duration of treatment, or every about 7 days, (iii) uveitis, wherein the duration of treatment is between about 4 weeks to about 12 weeks, and wherein the silicone hydrogel contact lens are replaced every about 5 to about 10 days throughout the duration of treatment, or every about 7 days, or (iv) corneal abrasion, wherein the duration of treatment is between about 5 days and about 3 weeks, and wherein the silicone hydrogel contact lens are replaced every about 5 to about 10 days throughout the duration of treatment, or every about 7 days.

27. (canceled)

28. (canceled)

29. (canceled)

30. The method of claim 24, wherein the ocular therapeutic agent is a drug selected from the group consisting of an antibiotic, an anti-inflammatory, an antihistamine, an antiviral agent, a cancer drug, an anesthetic, a cycloplegic, an anticholinergic, an antimuscarinic, a mydriatics, a lubricant agent, a hydrophilic agent, a decongestant, a vasoconstrictor, vasodilator, an immuno-suppressant, an immuno-modulating agent, an anti-glaucoma agent, an anti-infective, hyperosmolar agent, vitamins, growth factors, growth factor antagonists, sympathomimetics, an adrenergic agonist, an anti-cataract agent, an anti-hypertensive agent, an anti-macular degeneration agent, an ocular permeation enhancing agent, an anti-retinal disease agent, an anti-retinitis pigmentosa agent, an anti-diabetic retinopathy agent, an ocular myopia controlling agent, an ocular diagnostic agent, and combinations thereof.

31. (canceled)

32. The method of claim 30, wherein the drug is (i) an anti-inflammatory drug selected from the group consisting of triamcinolone acetonide, dexamethasone, dexamethasone sodium phosphate, and other corticosteroids, bromfenac sodium, diclofenac sodium and other NSAIDs, or (ii) an antibiotic selected from the group consisting of moxifloxacin or other quinolone or fluoroquinolone antibiotics, cefuroxime or other cephalosporin antibiotics, vancomycin or other glycopeptide antibiotics, and wherein the one or both eyes are further treated with an antibiotic with an optional long-acting SAID via intracameral irrigation or injection, subconjunctival or sub-Tenon's injection, or intravitreal injection and/or depot placement prior to the contacting of the contact lens delivery system to the one or both eyes of the mammal.

33. (canceled)

34. (canceled)

35. The method of claim 16, wherein the contact lens delivery system has a bioavailability that exceeds that of a conventional eye drop therapy for the same indication of use, and wherein the bioavailability of the contact lens delivery system is at least 5 times greater than a conventional eye drop therapy for the same indication of use.

36. The method of claim 16, wherein the contact lens delivery system provides a bioavailability in tear film (AUC₀₋₈ days) of at least about 140 μg week/mL or a bioavailability in tear film (AUC₀₋₂₄ hours) of at least about 20 μg day/mL.

37. The method of claim 36, wherein the contact lens delivery system provides a bioavailability in tear film (AUC₀₋₈ days) of at least about 1,000 μg week/mL or a bioavailability in tear film (AUC₀₋₂₄ hours) of at least about 100 μg day/mL when provided at a Cₘₐₓ concentration of at least about 200 μg/mL.

38. (canceled)

39. (canceled)

40. The method of claim 16, wherein the contact lens delivery system provides an average concentration of the at least one ocular therapeutic agent to the eye(s) of at least about 0.005 μg/mL per day, at least about 0.01 μg/mL per day, at least about 0.01 μg/mL per day, at least about 1 μg/mL per day, at least about 10 μg/mL per day, or at least about 100 μg/mL per day.
